(19) United States
(12) Patent Application Publication
(10) Pub. No.: US 2019/0360033 A1
(43) Pub. Date: Nov. 28, 2019

(54) METHODS FOR ASSESSING RISK USING MISMATCH AMPLIFICATION AND STATISTICAL METHODS

(71) Applicant: The Medical College of Wisconsin, Inc., Milwaukee, WI (US)

(72) Inventors: Karl Stamm, Wauwatosa, WI (US); Aoy Tomita Mitchell, Elm Grove, WI (US); Michael Mitchell, Elm Grove, WI (US)

(73) Assignee: The Medical College of Wisconsin, Inc., Milwaukee, WI (US)

(21) Appl. No.: 16/347,185

(22) PCT Filed: Nov. 2, 2017
(86) PCT No.: PCT/US17/59802

§ 371 Date: May 2, 2019

Related U.S. Application Data
(60) Provisional application No. 62/416,696, filed on Nov. 2, 2016.
(60) Provisional application No. 62/546,789, filed on Aug. 17, 2017.

(51) Int. Cl. C12Q 1/6858; G16B 20/00

(52) U.S. Cl. CPC C12Q 1/6858; C12Q 2535/125; G16B 20/00

(57) ABSTRACT

This invention relates to methods and compositions for assessing an amount of non-native nucleic acids in a sample, such as from a subject. The methods and compositions provided herein can be used to determine risk of a condition, such as transplant rejection, in subject.

### RELATED APPLICATIONS

This application claims the benefit under 35 U.S.C. § 119(e) of the filing date of U.S. Provisional Application 62/416,696, filed Nov. 2, 2016, and U.S. Provisional Application 62/546,789, filed Aug. 17, 2017, the contents of each of which are incorporated by reference herein in their entirety.

### FIELD OF THE INVENTION

This invention relates to methods and compositions for assessing an amount of non-native nucleic acids in a sample from a subject. The methods and compositions provided herein can be used to determine risk of a condition, such as transplant rejection. This invention further relates to methods and compositions for assessing the amount of non-native cell-free deoxyribonucleic acid (non-native cell-free DNA, such as donor-specific cell-free DNA) using multiplexed optimized mismatch amplification (MOMA).

### BACKGROUND OF THE INVENTION

The ability to detect and quantify non-native nucleic acids in a sample may permit the early detection of a condition, such as transplant rejection. Current methods for quantitative analysis of heterogeneous nucleic acid populations (e.g., a mixture of native and non-native nucleic acids), however, are limited.

### SUMMARY OF INVENTION

The present disclosure is based, at least in part on the surprising discovery that multiplexed optimized mismatch amplification can be used to quantify low frequency non-native nucleic acids in samples from a subject. Multiplexed optimized mismatch amplification embraces the design of primers that can include a 3′ penultimate mismatch for the amplification of a specific sequence but a double mismatch relative to an alternate sequence. Amplification with such primers can permit the quantitative determination of amounts of non-native nucleic acids in a sample, even where the amount of non-native nucleic acids are, for example, below 1%, or even 0.5%, in a heterogeneous population of nucleic acids.

Provided herein are methods, compositions, kits and reports related to such optimized amplification. The methods, compositions, kits and reports can be any one of the methods, compositions, kits and reports, respectively, provided herein, including any one of those of the Examples and Figures.

In one aspect, a method of assessing an amount of non-native nucleic acids in a sample from a subject, the sample comprising non-native and native nucleic acids is provided. The method may comprise obtaining results from a mismatch amplification-based quantification assay, and determining an amount of the non-native nucleic acids in the sample based on the results, wherein the determining comprises averaging the results to determine the amount, and the averaging is taking the median.

In another aspect, a method of assessing an amount of non-native nucleic acids in a sample from a subject, the sample comprising non-native and native nucleic acids, comprising obtaining results from a mismatch amplification-based quantification assay, and determining an amount of the non-native nucleic acids in the sample based on the results, wherein the determining comprises analyzing the results using a robust standard deviation and/or robust coefficient of variation is provided.

In another aspect, a method of assessing an amount of non-native nucleic acids in a sample from a subject, the sample comprising non-native and native nucleic acids, comprising obtaining results from a mismatch amplification-based quantification assay, and determining an amount of the non-native nucleic acids in the sample based on the results, wherein the determining comprises analyzing the results using a discordance value is provided.

In one embodiment of any one of the methods provided herein, the determining comprises or the method further comprises analyzing the results using a robust standard deviation and/or robust coefficient of variation.

In one embodiment of any one of the methods provided herein, the determining comprises or the method further comprises analyzing the results using a discordance value.

In another aspect, a method of assessing a risk in a subject based on one or more amounts of non-native nucleic acids in one or more samples from a subject, the sample(s) comprising non-native and native nucleic acids, comprising obtaining one or more amounts of non-native nucleic acids in one or more samples from a subject, which amounts are determined from one or more mismatch amplification-based quantification assays, each as defined in any one of such an assay provided herein, and assessing a risk based on the amount(s) of non-native nucleic acids.

In one embodiment of any one of the methods provided herein, the amount(s) are obtained from or provided in a report.

In one embodiment of any one of the methods provided herein, the amount(s) are the ratio or percentage of non-native nucleic acids to native nucleic acids or total nucleic acids. In one embodiment of any one of the methods provided herein, the amount(s) of the native or total nucleic acids are also determined.

In one embodiment of any one of the methods provided herein, the mismatch amplification-based quantitative assay comprises, for each of a plurality of single nucleotide variant (SNV) targets, nucleic acid amplification, such as a polymerase chain reaction (PCR), on a sample, or portion thereof, with at least one primer pair, wherein the at least one primer pair comprises a forward primer and a reverse primer, wherein the at least one primer pair comprises a primer with a 3′ mismatch (e.g., penultimate mismatch) relative to one sequence (e.g., allele) of the SNV target but a 3′ double mismatch relative to another sequence (e.g., allele) of the SNV target and specifically amplifies the one sequence (e.g., allele) of the SNV target.

In one embodiment of any one of the methods provided herein, the mismatch amplification-based quantitative assay further comprises, for each SNV target, nucleic acid amplification with at least one another primer pair, wherein the at least one another primer pair comprises a forward primer and a reverse primer, wherein the at least one another primer pair specifically amplifies another sequence (e.g., allele) of the SNV target.

In one embodiment of any one of the methods provided herein, the mismatch amplification-based quantitative assay comprises, for each of a plurality of single nucleotide variant (SNV) targets, nucleic acid amplification, such as a PCR, on a sample, or portion thereof, with at least two primer pairs, wherein each primer pair comprises a forward primer and a reverse primer, wherein one of the at least two primer pairs comprises a 3′ mismatch (e.g., penultimate) relative to one sequence (e.g., allele) of the SNV target but a 3′ double mismatch relative to another sequence (e.g., allele) of the SNV target and specifically amplifies the one sequence (e.g., allele) of the SNV target, and another of the at least two primer pairs specifically amplifies the another sequence (e.g., allele) of the SNV target.

In one embodiment of any one of the methods provided herein, the mismatch amplification-based quantitative assay comprises, for a plurality of SNV targets, for each such SNV target, nucleic acid amplification, such as PCR, of the sample with at least one primer pair as provided herein, such as at least two primer pairs, wherein each primer pair comprises a forward primer and a reverse primer, selecting informative results based on the genotype of the native nucleic acids and/or non-native nucleic acids.

In one embodiment of any one of the methods provided herein, the method may comprise determining the amount of the non-native nucleic acids in the sample based on the informative results.

In one embodiment of any one of the methods provided herein, the mismatch amplification-based quantitative assay further comprises identifying the plurality of SNV targets. In one embodiment of any one of the methods provided herein, the mismatch amplification-based quantitative assay further comprises inferring the genotype of the non-native nucleic acids.

In one embodiment of any one of the methods provided herein, the determining the amount comprises averaging, such as taking the median. In one embodiment of any one of the methods provided herein, the amount is based on an average, such as the median, of the results, such as the informative results.

In one embodiment of any one of the methods provided herein, the determining comprises or the method further comprises analyzing the results using Robust Statistics. In one embodiment of any one of the methods provided, the results can be analyzed with a Standard Deviation, such as a Robust Standard Deviation, and/or Coefficient of Variation, such as a Robust Coefficient of Variation, or % Coefficient of Variation, such as a % Robust Coefficient of Variation. In one embodiment of any one of the methods provided herein, the amount is based at least in part on, or the method further comprises, analysis of the results using Robust Statistics. In one embodiment of any one of the methods provided, the analysis includes the use of a Standard Deviation, such as a Robust Standard Deviation, and/or Coefficient of Variation, such as a Robust Coefficient of Variation, or % Coefficient of Variation, such as a % Robust Coefficient of Variation.

In one embodiment of any one of the methods provided herein, the determining comprises or the method further comprises analyzing the results using a discordance value. In one embodiment of any one of the methods provided, the results can be analyzed with a discordance value. In one embodiment of any one of the methods provided herein, the amount is based at least in part on, or the method further comprises, analysis of the results using a discordance value. In one embodiment of any one of the methods provided, the analysis includes the use of a discordance value.

In one embodiment of any one of the methods provided herein, the mismatch amplification-based quantitative assay comprises nucleic acid amplification, such as a PCR, for each of a plurality of SNV targets, performed on a sample, or portion thereof, with at least one primer pair, such as at least two primer pairs, wherein each primer pair comprises a forward primer and a reverse primer, wherein one of the at least one, such as at least two, primer pair, comprises a 3′ mismatch (e.g., penultimate) relative to one sequence (e.g., allele) of the SNV target but a 3′ double mismatch relative to another sequence (e.g., allele) of the SNV target and specifically amplifies the one sequence (e.g., allele) of the SNV target and a determination of informative results based on the native genotype and/or a prediction of the likely non-native genotype.

In one embodiment of any one of the methods provided herein, the mismatch amplification-based quantitative assay further comprises nucleic acid amplification, such as PCR, with at least one another primer pair for each SNV target. In one embodiment of any one of the methods provided herein, the at least one another primer pair comprises a 3′ mismatch (e.g., penultimate) relative to another sequence (e.g., allele) of the SNV target but a 3′ double mismatch relative to the one sequence (e.g., allele) of the SNV target and specifically amplifies the another sequence (e.g., allele) of the SNV target.

In one embodiment of any one of the methods provided herein, the method further comprises assessing the amount of non-native nucleic acids based on the amplification results. In one embodiment of any one of the methods provided herein, the results are informative results.

In one embodiment of any one of the methods provided herein, the mismatch amplification-based quantitative assay further comprises selecting informative results of the amplifications, such as PCR amplifications. In one embodiment of any one of the methods provided, the selected informative results are averaged, such as a median average. In one embodiment of any one of the methods provided herein, the method further comprises further analyzing the results with Robust Statistics. In one embodiment of any one of the methods provided, the results can be further analyzed with a Standard Deviation, such as a Robust Standard Deviation, and/or Coefficient of Variation, such as a Robust Coefficient of Variation, or % Coefficient of Variation, such as a % Robust Coefficient of Variation. In one embodiment of any one of the methods provided herein, the method further comprises analyzing the results with a discordance value. In one embodiment of any one of the methods provided, the results can be further analyzed with a discordance value.

In one embodiment of any one of the methods provided, the informative results of the nucleic acid amplifications, such as PCR, are selected based on the genotype of the non-native nucleic acids and/or native nucleic acids.

In one embodiment of any one of the methods provided, the method further comprises obtaining the genotype of the non-native nucleic acids and/or native nucleic acids.

In one embodiment of any one of the methods provided herein, the mismatch amplification-based quantitative assay further comprises selecting informative results based on the native genotype and/or prediction of the likely non-native genotype. In one embodiment of any one of the methods provided herein, when the genotype of the non-native nucleic acids is not known or obtained, the mismatch amplification-based quantitative assay further comprises assessing results based on a prediction of the likely non-native genotype. In one embodiment of any one of the methods provided, the assessing or prediction is performed with an expectation-maximization algorithm. In one embodiment of any one of the methods provided, expectation-maximization is used to predict the likely non-native genotype.

In one embodiment of any one of the methods provided, maximum likelihood is used to calculate the amount of non-native nucleic acids.

In one embodiment of any one of the methods provided herein, the mismatch amplification-based quantitative assay further comprises obtaining the plurality of SNV targets.

In one embodiment of any one of the methods provided herein, the mismatch amplification-based quantitative assay further comprises obtaining the at least one, such as at least two primer pairs, for each of the plurality of SNV targets.

In one embodiment of any one of the methods provided herein, the mismatch amplification-based quantitative assay further comprises obtaining or providing the results. In one embodiment of any one of the methods provided, the results are informative results.

In one embodiment of any one of the methods provided herein, the method further comprises obtaining or providing the amount(s).

In one embodiment of any one of the methods provided herein, the results or amount(s) are provided in a report.

In one aspect, a report containing the results and/or amount(s) of any one of the methods provided herein is provided. In one embodiment of any one of the methods or reports provided, the results are informative results. In one embodiment of any one of the methods provided herein, the results are obtained from a report. In one embodiment of any one of the reports provided, the report is given in electronic form. In one embodiment of any one of the reports provided, the report is a hard copy. In one embodiment of any one of the reports provided, the report is given orally.

In one embodiment of any one of the methods, there is at least one primer pair, at least two primer pairs, at least three primer pairs, at least four primer pairs or more per SNV target. In one embodiment of any one of the methods provided, the plurality of SNV targets is at least 45, 48, 50, 55, 60, 65, 70, 75, 80, 85 or 90 or more. In one embodiment of any one of the methods provided, the plurality of SNV targets is at least 90, 95 or more targets. In one embodiment of any one of the methods provided, the plurality of SNV targets is less than 90, 95 or more targets. In one embodiment of any one of the methods provided, the plurality of SNV targets is less than 105 or 100 targets.

In one embodiment of any one of the methods provided, the mismatched primer(s) is/are the forward primer(s). In one embodiment of any one of the methods, the reverse primers for the primer pairs for each SNV target is the same.

In one embodiment of any one of the methods provided, the amount of non-native nucleic acids in the sample is at least 0.005%. In one embodiment of any one of the methods provided, the amount of non-native nucleic acids in the sample is at least 0.01%. In one embodiment of any one of the methods provided, the amount of non-native nucleic acids in the sample is at least 0.03%. In one embodiment of any one of the methods provided, the amount of non-native nucleic acids in the sample is at least 0.05%. In one embodiment of any one of the methods provided, the amount of non-native nucleic acids in the sample is at least 0.1%. In one embodiment of any one of the methods provided, the amount of non-native nucleic acids in the sample is at least 0.3%. In one embodiment of any one of the methods provided, the amount of non-native nucleic acids in the sample is less than 1.5%. In one embodiment of any one of the methods provided, the amount of non-native nucleic acids in the sample is less than 1.3%. In one embodiment of any one of the methods provided, the amount of non-native nucleic acids in the sample is less than 1%. In one embodiment of any one of the methods provided, the amount of non-native nucleic acids in the sample is less than 0.5%.

In one embodiment of any one of the methods provided, the sample comprises cell-free DNA sample and the amount is an amount of non-native cell-free DNA.

In one embodiment of any one of the methods provided, the subject is a transplant recipient, and the amount of non-native nucleic acids is an amount of donor-specific cell-free DNA.

In one embodiment of any one of the methods provided, the transplant recipient is a heart transplant recipient. In one embodiment of any one of the methods provided, the transplant recipient is a pediatric transplant recipient, such as a pediatric heart transplant recipient.

In one embodiment of any one of the methods provided, the amplifications, such as PCR, are real time PCR or digital PCR amplifications.

In one embodiment of any one of the methods provided, the method further comprises determining a risk in the subject based on the amount of non-native nucleic acids in the sample. In one embodiment of any one of the methods provided, the risk is a risk associated with a transplant. In one embodiment of any one of the methods provided, the risk associated with a transplant is risk of transplant rejection, an anatomical problem with the transplant or injury to the transplant. In one embodiment of any one of the methods provided herein, the injury to the transplant is initial or ongoing injury. In one embodiment of any one of the methods provided herein, the risk associated with the transplant is indicative of the severity of the injury.

In one embodiment of any one of the methods provided, the risk is increased if the amount of non-native nucleic acids is greater than a threshold value. In one embodiment of any one of the methods provided, the risk is decreased if the amount of non-native nucleic acids is less than a threshold value.

In one embodiment of any one of the methods provided, where the risk is the risk associated with the heart transplant rejection, the threshold value is 1%. In one embodiment of any one of the methods provided, where the risk is the risk associated with the heart transplant rejection, the threshold value is 1.3%.

In one embodiment of any one of the methods provided, the method further comprises selecting a treatment for the subject based on the amount of non-native nucleic acids.

In one embodiment of any one of the methods provided, the method further comprises treating the subject based on the amount of non-native nucleic acids.

In one embodiment of any one of the methods provided, the method further comprises providing information about a treatment to the subject based on the amount of non-native nucleic acids.

In one embodiment of any one of the methods provided, method further comprises monitoring or suggesting the monitoring of the amount of non-native nucleic acids in the subject over time.

In one embodiment of any one of the methods provided, the method further comprises assessing the amount of non-native nucleic acids in the subject at a subsequent point in time.

In one embodiment of any one of the methods provided, the method further comprises obtaining another sample from the subject, such as at a subsequent point in time, and performing a test on the sample, such as any one of the methods provided herein.

In one embodiment of any one of the methods provided, the method further comprises evaluating an effect of a treatment administered to the subject based on the amount of non-native nucleic acids.

In one embodiment of any one of the methods provided, the treatment is an anti-rejection therapy.

In one embodiment of any one of the methods provided, the treatment is an anti-infection therapy.

In one embodiment of any one of the methods provided, the method further comprises providing or obtaining the sample or a portion thereof.

In one embodiment of any one of the methods provided, the method further comprises extracting nucleic acids from the sample.

In one embodiment of any one of the methods provided, the sample comprises blood, plasma, or serum.

In one embodiment of any one of the methods or reports provided, the sample is obtained or is one that was obtained from the subject within 10 days of a heart transplant. In one embodiment of any one of the methods or reports provided herein, the sample is obtained or is one that was obtained from the subject within 14 hours of a surgery. In one embodiment of any one of the methods or reports provided herein, the sample is obtained or is one that was obtained from the subject within 24 hours of a surgery. In one embodiment of any one of the methods or reports provided herein, the surgery is a transplant surgery. In one embodiment of any one of the methods or reports provided herein, the sample is obtained or is one that was obtained from the subject within 14 hours of cross-clamp removal. In one embodiment of any one of the methods or reports provided herein, the sample is obtained or is one that was obtained from the subject within 24 hours of cross-clamp removal.

In one embodiment of any one of the methods provided herein, the amounts are determined or obtained on a weekly basis over time. In one embodiment of any one of the methods provided herein, the amounts are determined or obtained on a bi-weekly basis over time. In one embodiment of any one of the methods provided herein, the amounts are determined or obtained on a monthly basis over time.

In one embodiment, any one of the embodiments for the methods provided herein can be an embodiment for any one of the reports provided. In one embodiment, any one of the embodiments for the reports provided herein can be an embodiment for any one of the methods provided herein.

### BRIEF DESCRIPTION OF DRAWINGS

The accompanying drawings are not intended to be drawn to scale. The figures are illustrative only and are not required for enablement of the disclosure.

FIG. 1 provides an exemplary, non-limiting diagram of MOMA primers. In a polymerase chain reaction (PCR) assay, extension of the sequence containing SNV A is expected to occur, resulting in the detection of SNV A, which may be subsequently quantified. Extension of the SNV B, however, is not expected to occur due to the double mismatch.

FIG. 2 provides exemplary amplification traces.

FIG. 3 shows results from a reconstruction experiment demonstrating proof of concept.

FIG. 4 provides the percent cell-free DNA measured with plasma samples from transplant recipient patients. All data comes from patients who have had biopsies. Dark points denote rejection.

FIG. 5 provides further data from a method as provided herein on plasma samples. After transplant surgery, the donor percent levels drop off.

FIG. 6 demonstrates the use of expectation maximization to predict non-native donor genotype when unknown. Black=background, Green=half informative, Red=fully informative, Dashed line=first iteration, Solid line=second iteration, Final call=10%.

FIG. 7 demonstrates the use of expectation maximization to predict non-native donor genotype when unknown. Black=background, Green=half informative, Red=fully informative, Final call=5%.

FIG. 8 provides reconstruction experiment data demonstrating the ability to predict the non-native donor genotype when unknown. Data have been generated with a set of 95 SNV targets.

FIG. 9 provides the average background noise for 104 MOMA targets.

FIG. 10 provides further examples of the background noise for methods using MOMA.

FIGS. 11-30 illustrate the benefit of having the probe on the same strand as the mismatch primer in some embodiments.

FIG. 31 illustrates an example of a computer system with which some embodiments may operate.

### DETAILED DESCRIPTION OF THE INVENTION

Aspects of the disclosure relate to methods for the sensitive detection and/or quantification of non-native nucleic acids in a sample. Non-native nucleic acids, such as non-native DNA, may be present in individuals in a variety of situations including following organ transplantation. The disclosure provides techniques to detect, analyze and/or quantify non-native nucleic acids, such as non-native cell-free DNA concentrations, in samples obtained from a subject.

As used herein, “non-native nucleic acids” refers to nucleic acids that are from another source or are mutated versions of a nucleic acid found in a subject (with respect to a specific sequence). “Native nucleic acids”, therefore, are nucleic acids that are not from another source and are not mutated versions of a nucleic acid found in a subject (with respect to a specific sequence). In some embodiments, the non-native nucleic acid is non-native cell-free DNA. “Cell-free DNA” (or cf-DNA) is DNA that is present outside of a cell, e.g., in the blood, plasma, serum, etc. of a subject. Without wishing to be bound by any particular theory or mechanism, it is believed that cf-DNA is released from cells, e.g., via apoptosis of the cells. An example of non-native nucleic acids are nucleic acids that are from a donor of a transplant in a transplant recipient subject. As used herein, the compositions and methods provided herein can be used to determine an amount of cell-free DNA from a non-native source, such as DNA specific to a donor or donor-specific cell-free DNA (e.g., donor-specific cfDNA).

Provided herein are methods and compositions that can be used to measure nucleic acids with differences in sequence identity. In some embodiments, the difference in sequence identity is a single nucleotide variant (SNV); however, wherever a SNV is referred to herein any difference in sequence identity between native and non-native nucleic acids is intended to also be applicable. Thus, any one of the methods provided herein may be applied to native versus non-native nucleic acids where there is a difference in sequence identity. As used herein, “single nucleotide variant” refers to a nucleic acid sequence within which there is sequence variability at a single nucleotide. In some embodiments, the SNV is a biallelic SNV, meaning that there is one major allele and one minor allele for the SNV. In some embodiments, the SNV may have more than two alleles, such as within a population. In some embodiments, the SNV is a mutant version of a sequence, and the non-native nucleic acid refers to the mutant version, while the native nucleic acid refers to the non-mutated version (such as wild-type version). Such SNVs, thus, can be mutations that can occur within a subject and which can be associated with a disease or condition. Generally, a “minor allele” refers to an allele that is less frequent, such as in a population, for a locus, while a “major allele” refers to the more frequent allele, such as in a population. The methods and compositions provided herein can quantify nucleic acids of major and minor alleles within a mixture of nucleic acids even when present at low levels, in some embodiments.

The nucleic acid sequence within which there is sequence identity variability, such as a SNV, is generally referred to as a “target”. As used herein, a “SNV target” refers to a nucleic acid sequence within which there is sequence variability at a single nucleotide, such as in a population of individuals or as a result of a mutation that can occur in a subject and that can be associated with a disease or condition. The SNV target has more than one allele, and in preferred embodiments, the SNV target is biallelic. In some embodiments of any one of the methods provided herein, the SNV target is a SNP target. In some of these embodiments, the SNP target is biallelic. It has been discovered that non-native nucleic acids can be quantified even at extremely low levels by performing amplification-based quantitative assays, such as PCR assays with primers specific for SNV targets as provided herein. In some embodiments, the amount of non-native nucleic acids is determined by attempting amplification-based quantitative assays, such as quantitative PCR assays, with primers for a plurality of SNV targets.

A “plurality of SNV targets” refers to more than one SNV target where for each target there are at least two alleles. Preferably, in some embodiments, each SNV target is expected to be biallelic and a primer pair specific to each allele of the SNV target is used to specifically amplify nucleic acids of each allele, where amplification occurs if the nucleic acid of the specific allele is present in the sample. In some embodiments, the plurality of SNV targets are a plurality of sequences within a subject that can be mutated and that if so mutated can be indicative of a disease or condition in the subject. As used herein, one allele may be the mutated version of a target sequence and another allele is the non-mutated version of the sequence.

In some embodiments, the amplification-based quantitative assay, such as quantitative PCR, is performed with primer pairs for at least 40, 45, 50, 55, 60, 65, 70, 75, 80, 85, 90, 91, 92, 93, 94, 95 or more targets. In some embodiments, the quantitative assay is performed with primer pairs for fewer than 105, 104, 103, 102, 101, 100, 99, 98 or 97 targets. In some embodiments, sufficient informative results are obtained with primer pairs for between 40-105, 45-105, 50-105, 55-105, 60-105, 65-105, 70-105, 75-105, 80-105, 85-105, 90-105, 90-104, 90-103, 90-102, 90-101, 90-100, 90-99, 91-99, 92-99, 93, 99, 94-99, 95-99, or 90-95 targets. In some embodiments, sufficient informative results are obtained with primer pairs for between 40-99, 45-99, 50-99, 55-99, 60-99, 65-99, 70-99, 75-99, 80-99, 85-99, 90-99, 90-99, 90-98, 90-97 or 90-96 targets. In still other embodiments, sufficient informative results are obtained with primer pairs for between 40-95, 45-95, 50-95, 55-95, 60-95, 65-95, 70-95, 75-95, 80-95, 85-95, or 90-95 targets. In still other embodiments, sufficient informative results are obtained with primer pairs for between 40-90, 45-90, 50-90, 55-90, 60-90, 65-90, 70-90, 75-90, 80-90, or 85-90 targets. In still other embodiments, sufficient informative results are obtained with primer pairs for between 40-85, 45-85, 50-85, 55-85, 60-85, 65-85, 70-85, 75-85, or 80-85 targets. In still other embodiments, sufficient informative results are obtained with primer pairs for between 40-80, 45-80, 50-80, 55-80, 60-80, 65-80, 70-80, or 75-80 targets. In still other embodiments, sufficient informative results are obtained with primer pairs for between 40-75, 45-75, 50-75, 55-75, 60-75, 65-75, or 70-75 targets.

“Informative results” as provided herein are the results that can be used to quantify the level of non-native or native nucleic acids in a sample. Generally, informative results exclude the results where the native nucleic acids are heterozygous for a specific SNV target as well as “no call” or erroneous call results. From the informative results, allele percentages can be calculated using standard curves, in some embodiments of any one of the methods provided. In some embodiments of any one of the methods provided, the amount of non-native and/or native nucleic acids represents an average across informative results for the non-native and/or native nucleic acids, respectively. In some embodiments of any one of the methods provided herein, this average is given as an absolute amount or as a percentage. Preferably, in some embodiments of any one of the methods provided herein, this average is the median. In other embodiments of any one of the methods provided herein, the average is a trimmed mean. As used herein, the “trimmed mean” refers to the removal of the lowest reporting targets (such as the two lowest) in combination with the highest of the reporting targets (such as the two highest). In still other embodiments of any one of the methods provided herein, the average is the mean.

In some embodiments of any one of the methods provided herein, the method can further comprise the use of Robust Statistics (e.g., BD FACSDiva™ Software) to analyze the results. In some of such embodiments, the use of such statistics can be done at the end as a quality check of the results. In some of such embodiments, the statistics may indicate a sample may need to be rerun or some results should be discarded. In some embodiments, any one of the methods provided herein can include a step whereby a Standard Deviation, such as a Robust Standard Deviation (rSD), and/or a Coefficient of Variation, such as a Robust Coefficient of Variation (rCV), or % Coefficient of Variation, such as a % Robust Coefficient of Variation, can be calculated.

As used herein, the Robust SD is based upon the deviation of individual data points to the median of the population. It can be calculated as:

rSD=(Median of {|Xᵢ−Medianₓ|})×1.4826

The value 1.4826 is a constant factor that adjusts the resulting robust value to the equivalent of a normal population distribution. Thus, for a normally distributed population, the SD and the rSD are equal.

Similarly, the Robust CV and percent Robust CV can be calculated as:

rCV=rSD/Medianₓ and % rCV=rSD/Medianₓ×100%, respectively

Thus, in any one of the methods provided herein the final amounts can be determined at least in part on an analysis of the results using a Standard Deviation, such as rSD, and/or a Coefficient of Variation, such as rCV, or % Coefficient of Variation, such as % rCV.

In some embodiments of any one of the methods provided herein, the method can further comprise the use of a discordance value (dQC). For example, the average minor allele proportion of recipient homozygous and non-informative targets can be evaluated in order to safeguard against sample mixups and contamination. These should theoretically read nearly zero percent, subject to non-specificity allelic noise. If a sample-swap had occurred during collection or processing, the wrong recipient genotypes are used, the dQC can immediately flag up to 50 or 100% readings at presumed non-informative targets. The dQC can also captures sample contamination and possibly genomic instability. Generally, healthy samples will have a dQC below 0.5%.

The amount, such as ratio or percentage, of non-native nucleic acids may be determined with the quantities of the major and minor alleles as well as the genotype of the native and/or non-native nucleic acids. For example, results where the native nucleic acids are heterozygous for a specific SNV target can be excluded with knowledge of the native genotype. Further, results can also be assessed with knowledge of the non-native genotype. In some embodiments of any one of the methods provided herein, where the genotype of the native nucleic acids is known but the genotype of the non-native nucleic acids is not known, the method may include a step of predicting the likely non-native genotype or determining the non-native genotype by sequencing. Further details for such methods are provided elsewhere herein such as in the Examples. In some embodiments of any one of the methods provided herein, the alleles can be determined based on prior genotyping of the native nucleic acids of the subject and/or the nucleic acids not native to the subject (e.g., of the recipient and donor, respectively). Methods for genotyping are well known in the art. Such methods include sequencing, such as next generation, hybridization, microarray, other separation technologies or PCR assays. Any one of the methods provided herein can include steps of obtaining such genotypes.

“Obtaining” as used herein refers to any method by which the respective information or materials can be acquired. Thus, the respective information can be acquired by experimental methods, such as to determine the native genotype. Respective materials can be created, designed, etc. with various experimental or laboratory methods, in some embodiments. The respective information or materials can also be acquired by being given or provided with the information, such as in a report, or materials. Materials may be given or provided through commercial means (i.e., by purchasing), in some embodiments.

Reports may be in oral, written (or hard copy) or electronic form, such as in a form that can be visualized or displayed. In some embodiments, the “raw” results for each assay as provided herein are provided in a report, and from this report, further steps can be taken to determine the amount of non-native nucleic acids in the sample. These further steps may include any one or more of the following, selecting informative results, obtaining the native and/or non-native genotype, calculating allele percentages for informative results for the native and non-native nucleic acids, averaging the allele percentages, etc. In other embodiments, the report provides the amount of non-native nucleic acids in the sample. From the amount, in some embodiments, a clinician may assess the need for a treatment for the subject or the need to monitor the subject, such as the amount of the non-native nucleic acids later in time. Accordingly, in any one of the methods provided herein, the method can include assessing the amount of non-nucleic acids in the subject at another point in time. Such assessing can be performed with any one of the methods provided herein.

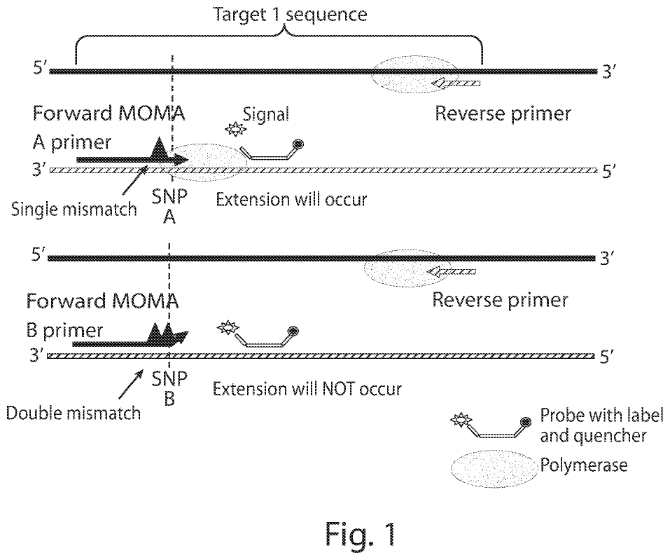

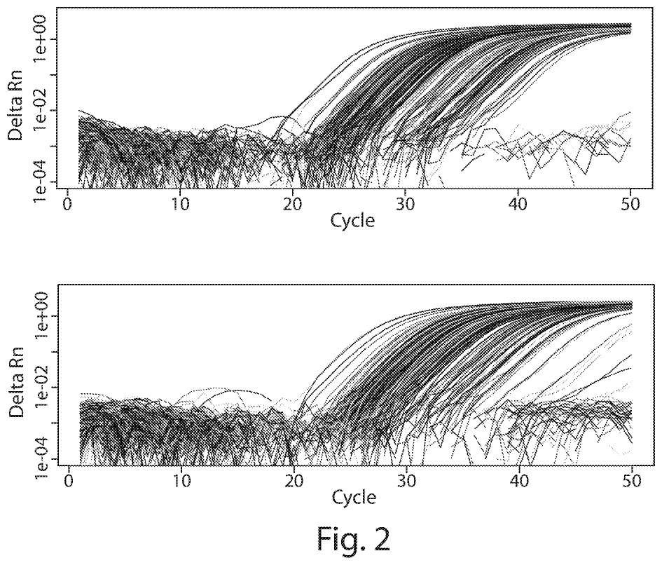

The amplification-based quantitative assays as provided herein make use of multiplexed optimized mismatch amplification (MOMA). Primers for use in such assays may be obtained, and any one of the methods provided herein can include a step of obtaining one or more primer pairs for performing the quantitative assays. Generally, the primers possess unique properties that facilitate their use in quantifying amounts of nucleic acids. For example, a forward primer of a primer pair can be mismatched at a 3′ nucleotide (e.g., penultimate 3′ nucleotide). In some embodiments of any one of the methods provided, this mismatch is at a 3′ nucleotide but adjacent to the SNV position. In some embodiments of any one of the methods provided, the mismatch positioning of the primer relative to a SNV position is as shown in FIG. 1. Generally, such a forward primer even with the 3′ mismatch to produce an amplification product (in conjunction with a suitable reverse primer) in an amplification reaction, thus allowing for the amplification and resulting detection of a nucleic acid with the respective SNV. If the particular SNV is not present, and there is a double mismatch with respect to the other allele of the SNV target, an amplification product will generally not be produced. Preferably, in some embodiments of any one of the methods provided herein, for each SNV target a primer pair is obtained whereby specific amplification of each allele can occur without amplification of the other allele(s). “Specific amplification” refers to the amplification of a specific allele of a target without substantial amplification of another nucleic acid or without amplification of another nucleic acid sequence above background or noise. In some embodiments, specific amplification results only in the amplification of the specific allele.

In some embodiments of any one of the methods provided herein, for each SNV target that is biallelic, there are two primer pairs, each specific to one of the two alleles and thus have a single mismatch with respect to the allele it is to amplify and a double mismatch with respect to the allele it is not to amplify (again if nucleic acids of these alleles are present). In some embodiments of any one of the methods provided herein, the mismatch primer is the forward primer. In some embodiments of any one of the methods provided herein, the reverse primer of the two primer pairs for each SNV target is the same.

These concepts can be used in the design of primer pairs for any one of the methods provided herein. It should be appreciated that the forward and reverse primers are designed to bind opposite strands (e.g., a sense strand and an antisense strand) in order to amplify a fragment of a specific locus of the template. The forward and reverse primers of a primer pair may be designed to amplify a nucleic acid fragment of any suitable size to detect the presence of, for example, an allele of a SNV target according to the disclosure. Any one of the methods provided herein can include one or more steps for obtaining one or more primer pairs as described herein.

It should be appreciated that the primer pairs described herein may be used in a multiplex PCR assay. Accordingly, in some embodiments of any one of the methods provided herein, the primer pairs are designed to be compatible with other primer pairs in a PCR reaction. For example, the primer pairs may be designed to be compatible with at least 2, at least 5, at least 10, at least 20, at least 30, at least 40, etc. other primer pairs in a PCR reaction. As used herein, primer pairs in a PCR reaction are “compatible” if they are capable of amplifying their target in the same PCR reaction. In some embodiments, primer pairs are compatible if the primer pairs are inhibited from amplifying their target DNA by no more than 1%, no more than 2%, no more than 5%, no more than 10%, no more than 15%, no more than 20%, no more than 25%, no more than 30%, no more than 35%, no more than 40%, no more than 45%, no more than 50%, or no more than 60% when multiplexed in the same PCR reaction. Primer pairs may not be compatible for a number of reasons including, but not limited to, the formation of primer dimers and binding to off-target sites on a template that may interfere with another primer pair. Accordingly, the primer pairs of the disclosure may be designed to prevent the formation of dimers with other primer pairs or limit the number of off-target binding sites. Exemplary methods for designing primers for use in a multiplex PCR assay are known in the art or are otherwise described herein.

In some embodiments, the primer pairs described herein are used in a multiplex PCR assay to quantify an amount of non-native nucleic acids. Accordingly, in some embodiments of any one of the methods provided herein, the primer pairs are designed to detect genomic regions that are diploid, excluding primer pairs that are designed to detect genomic regions that are potentially non-diploid. In some embodiments of any one of the methods provided herein, the primer pairs used in accordance with the disclosure do not detect repeat-masked regions, known copy-number variable regions, or other genomic regions that may be non-diploid.

As mentioned above, in some embodiments, any one of the methods provided herein may include steps of a “mismatch amplification method” or “mismatch amplification-based quantitative assay” or the like in order to determine a value for an amount of specific cell-free nucleic acids (such as DNA). In some embodiments of any one of the methods provided herein, the “mismatch amplification-based quantitative assay” is any quantitative assay whereby nucleic acids are amplified with the MOMA primers as described herein, and the amounts of the nucleic acids can be determined. Such methods comprise multiple amplifications from multiple SNV targets. Such methods include the methods of PCT Application No. PCT/US2016/030313, and any one of the methods provided herein may include the steps of any one of the methods described in PCT Application No. PCT/US2016/030313, and such methods and steps are incorporated herein by reference. In some embodiments of any one of the methods provided herein, such results of the multiple amplifications may be used to determine an amount of non-native nucleic acids in a sample by using one or more statistical methods, including the median, robust standard deviation, robust coefficient of variation, and discordance value. In some embodiments of any one of the methods provided herein, the quantitative assays are quantitative PCR assays. Quantitative PCR include real-time PCR, digital PCR, TAQMAN™, etc. In some embodiments of any one of the methods provided herein the PCR is “real-time PCR”. Such PCR refers to a PCR reaction where the reaction kinetics can be monitored in the liquid phase while the amplification process is still proceeding. In contrast to conventional PCR, real-time PCR offers the ability to simultaneously detect or quantify in an amplification reaction in real time. Based on the increase of the fluorescence intensity from a specific dye, the concentration of the target can be determined even before the amplification reaches its plateau.

The use of multiple probes can expand the capability of single-probe real-time PCR. Multiplex real-time PCR uses multiple probe-based assays, in which each assay can have a specific probe labeled with a unique fluorescent dye, resulting in different observed colors for each assay. Real-time PCR instruments can discriminate between the fluorescence generated from different dyes. Different probes can be labeled with different dyes that each have unique emission spectra. Spectral signals can be collected with discrete optics, passed through a series of filter sets, and collected by an array of detectors. Spectral overlap between dyes may be corrected by using pure dye spectra to deconvolute the experimental data by matrix algebra.

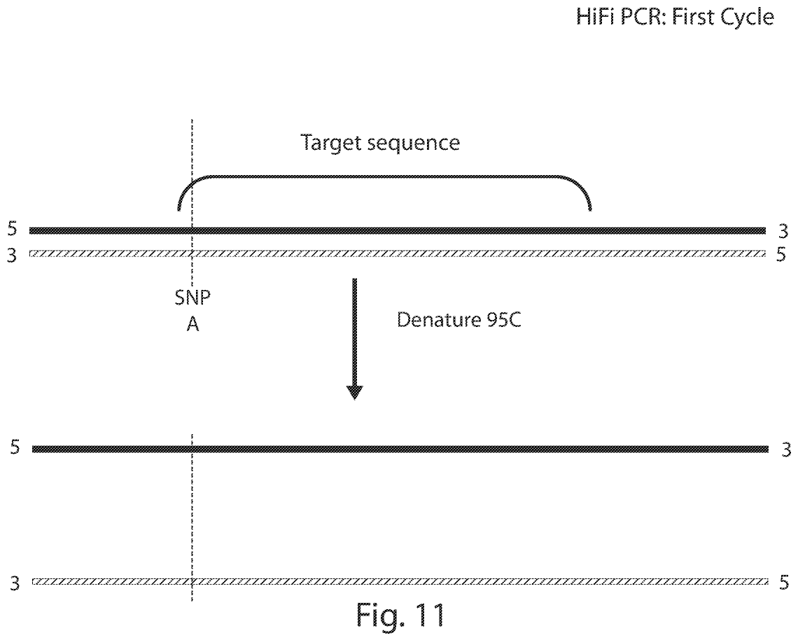

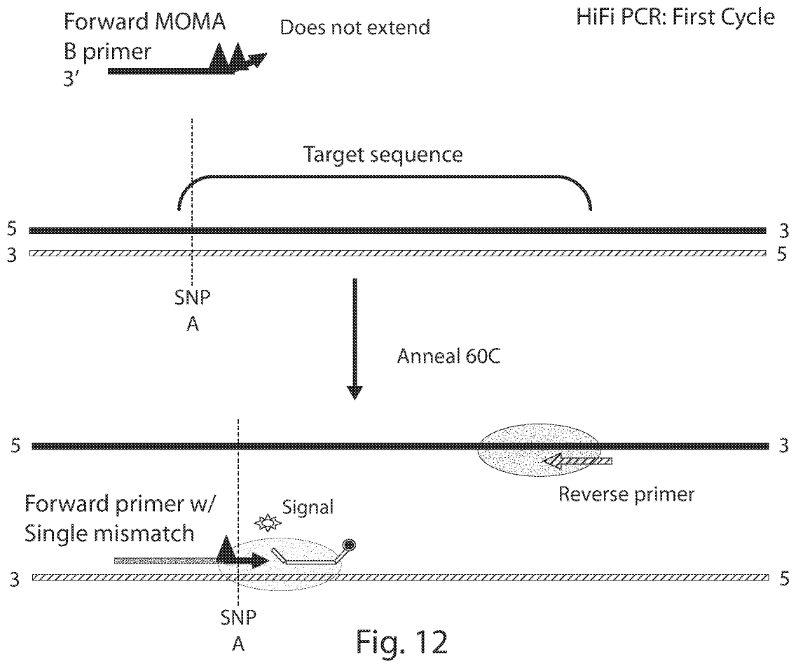

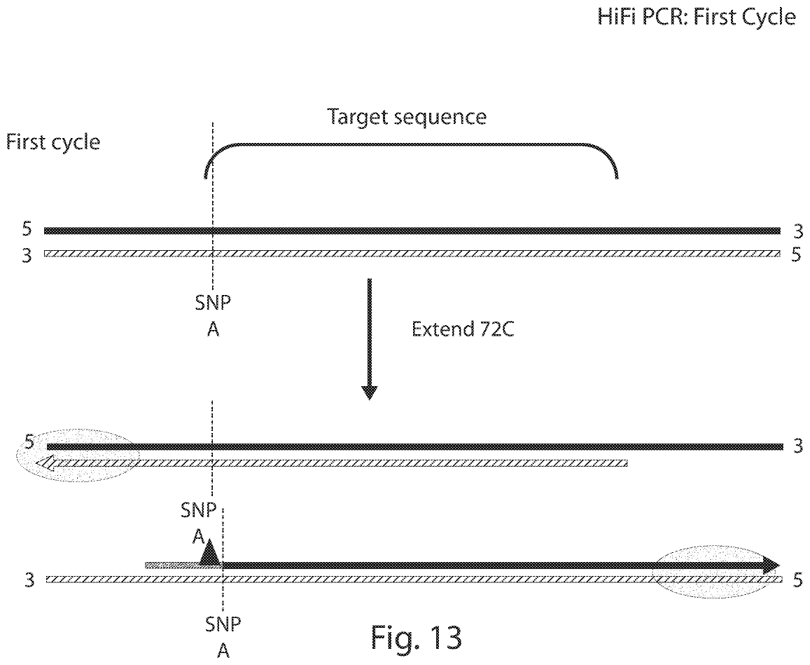

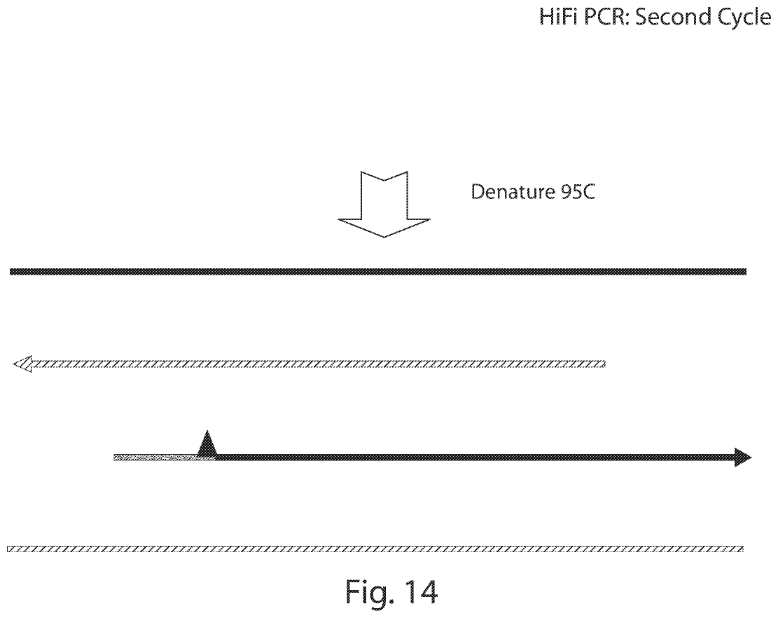

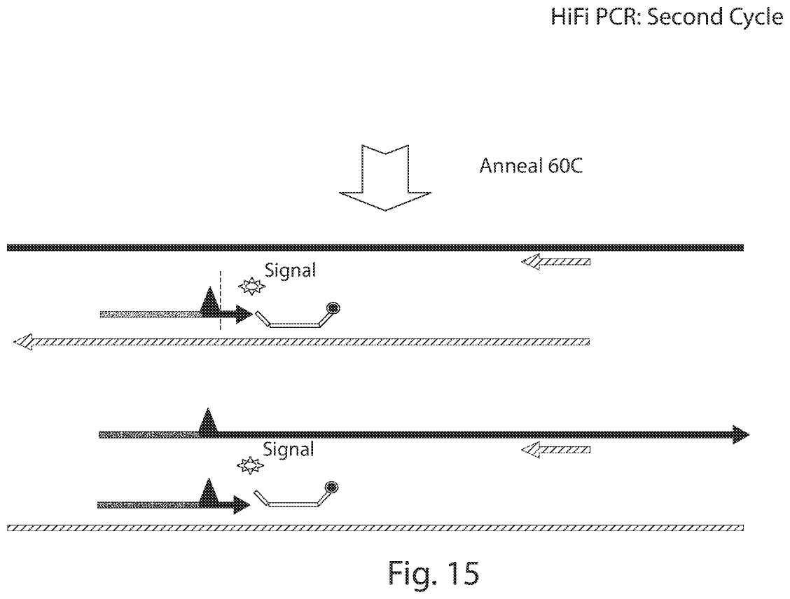

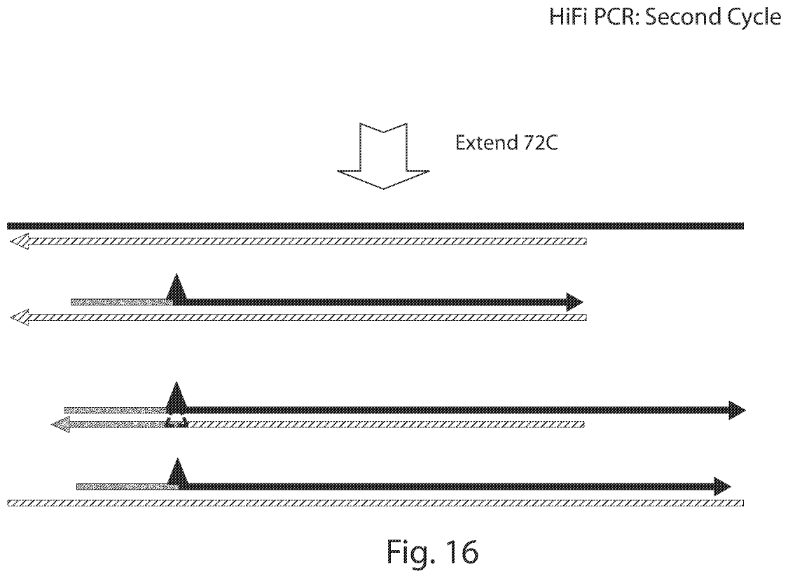

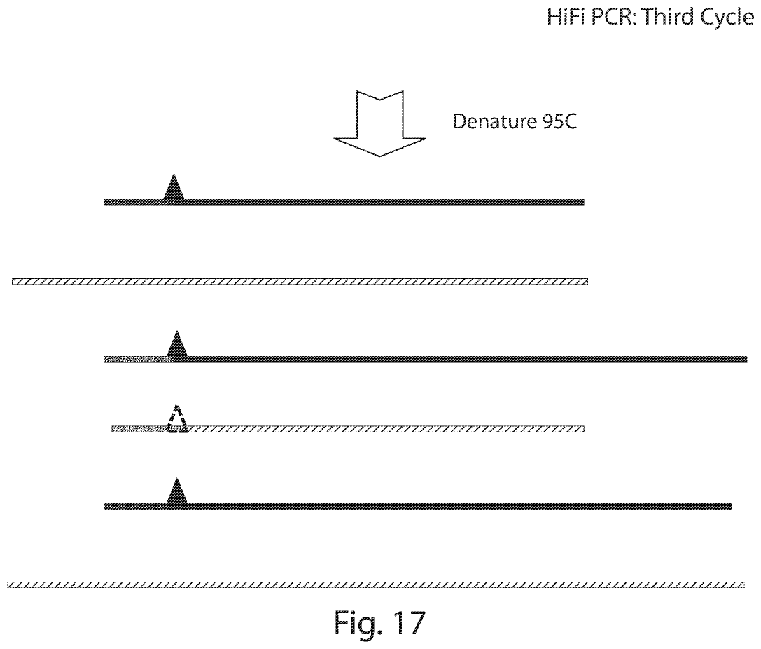

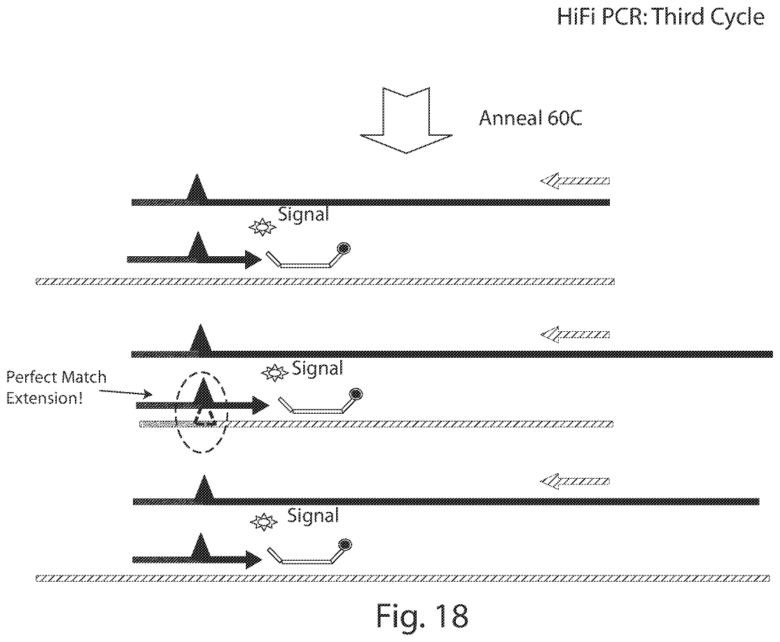

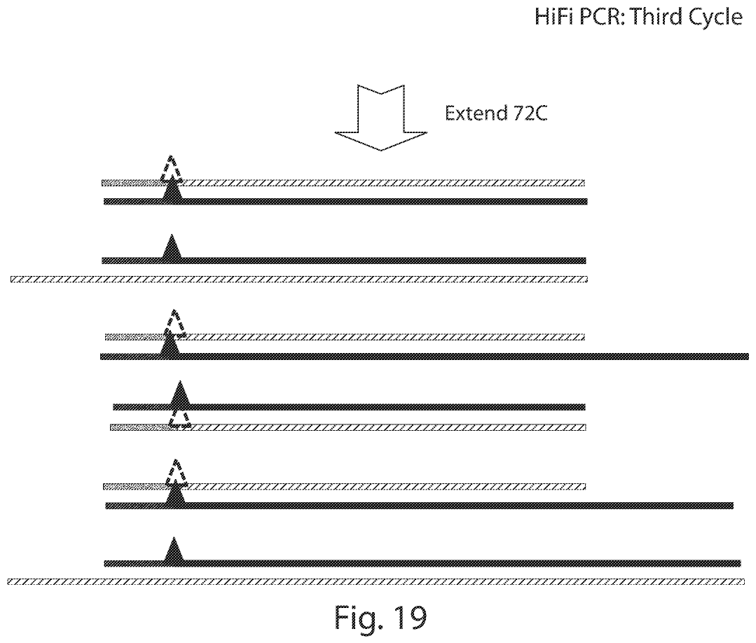

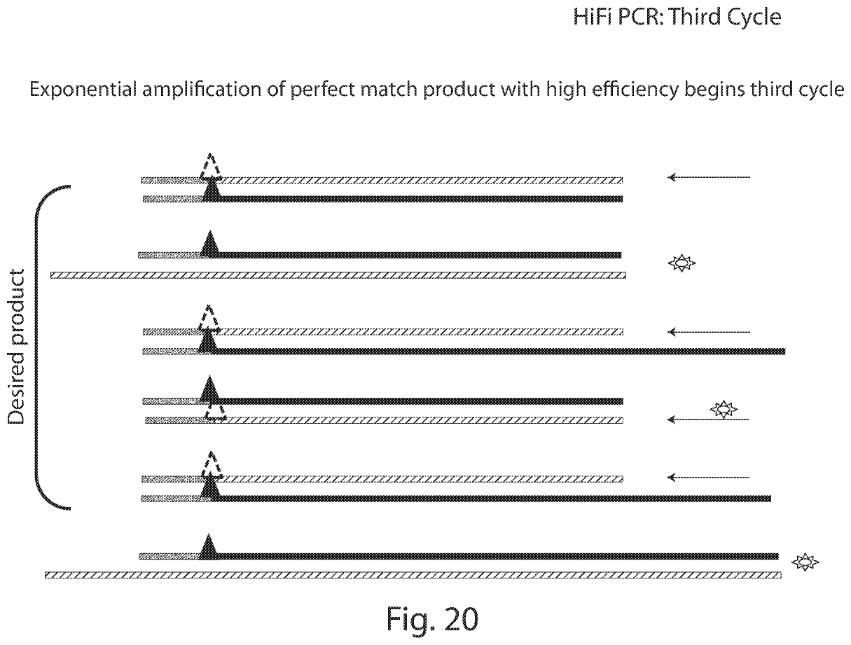

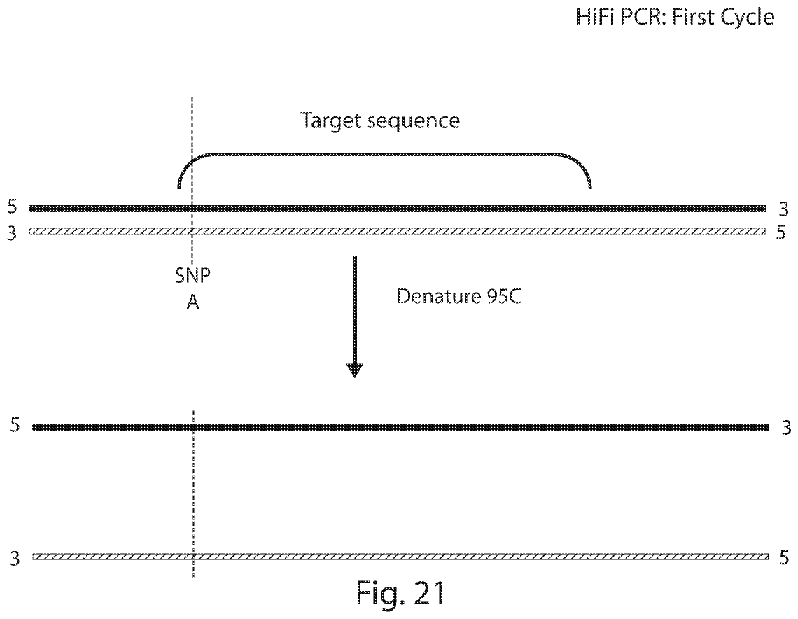

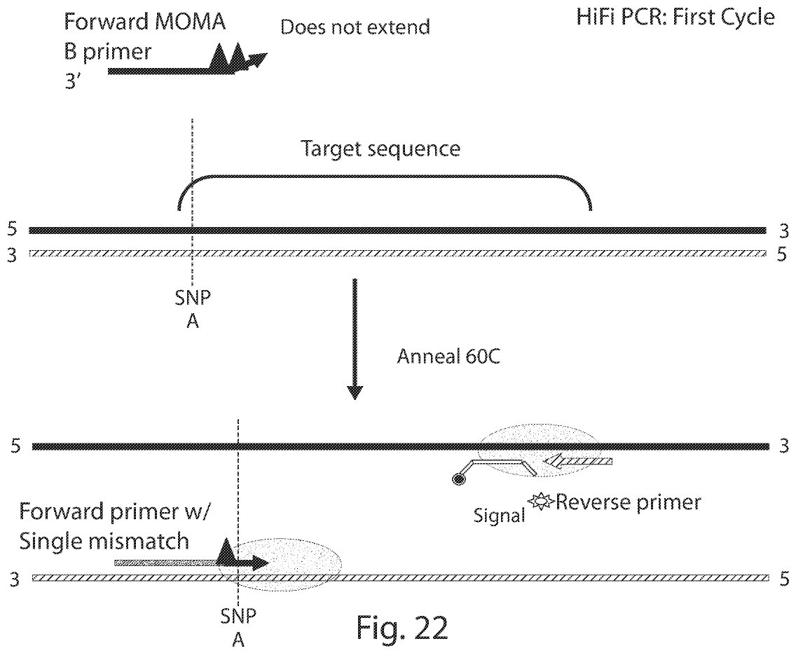

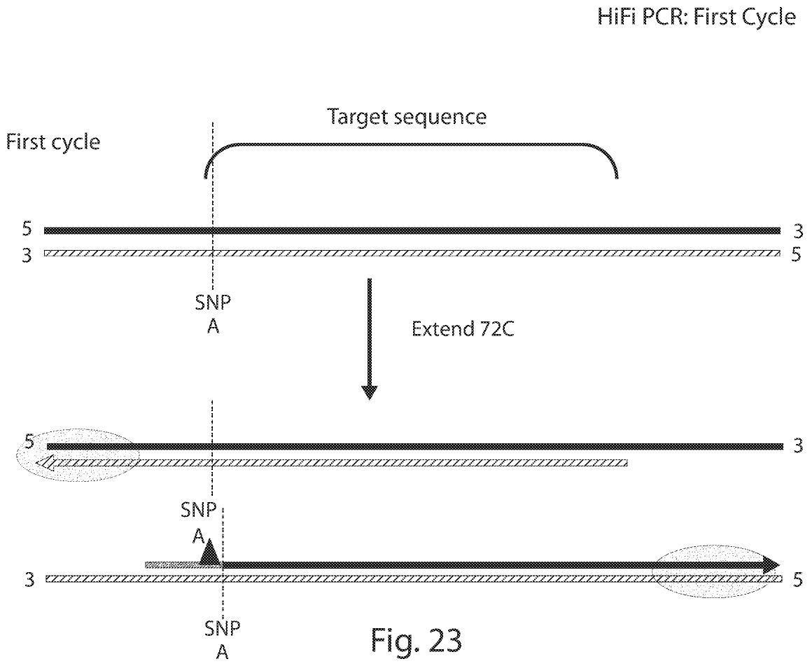

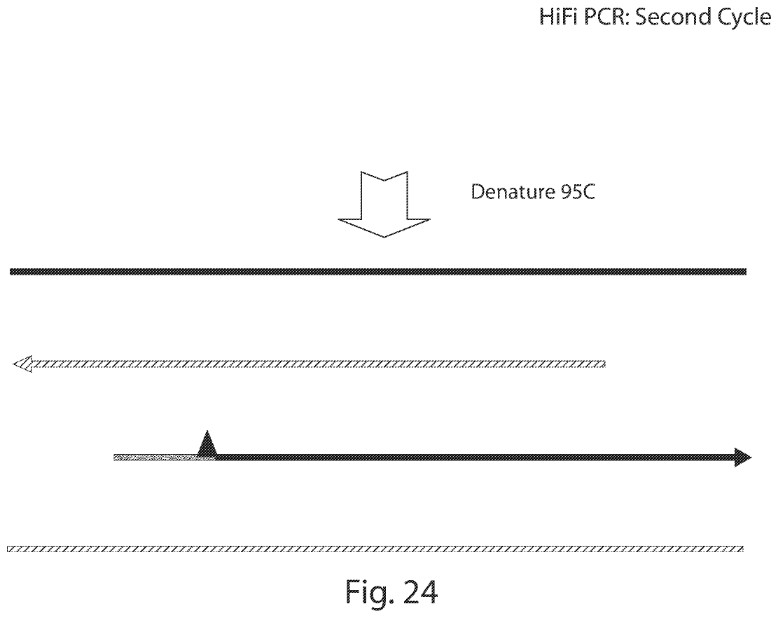

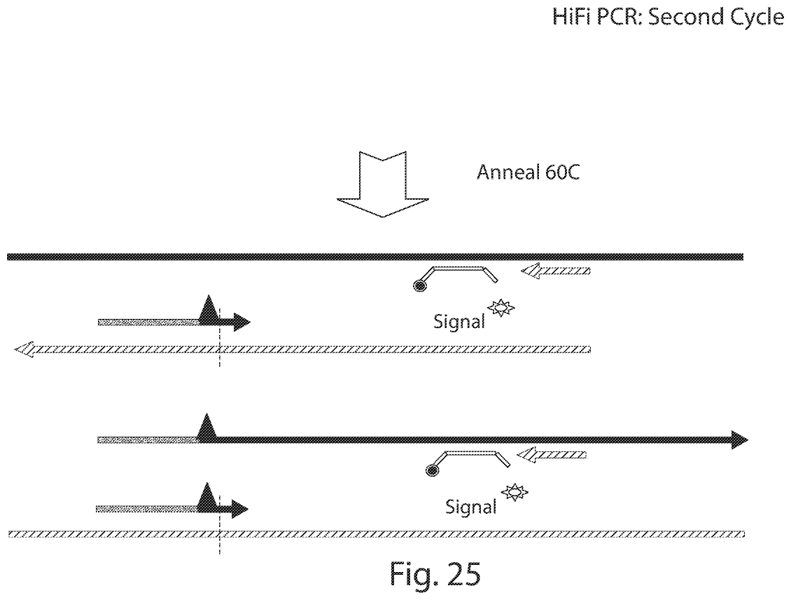

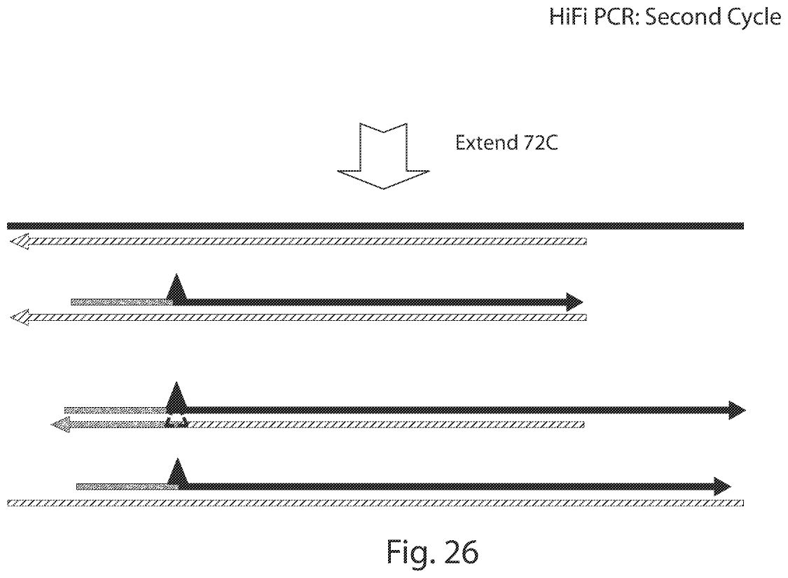

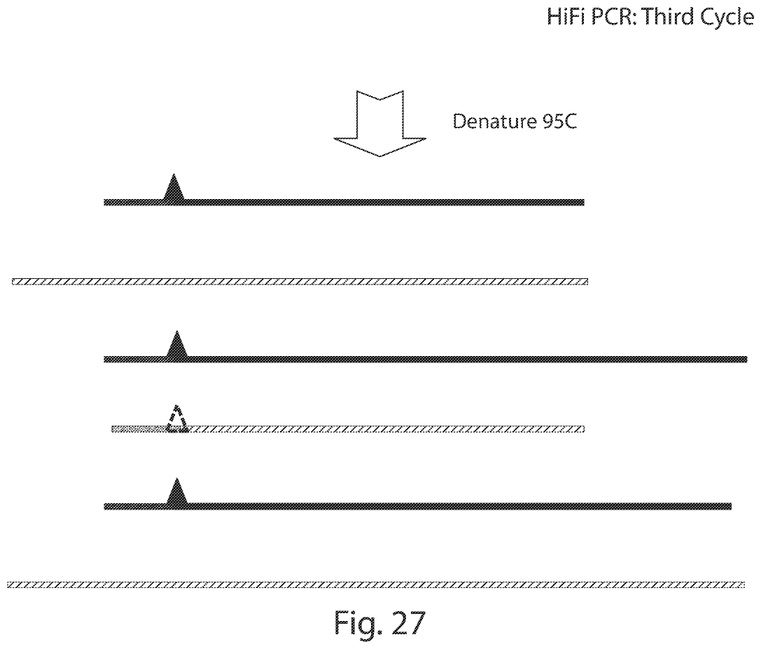

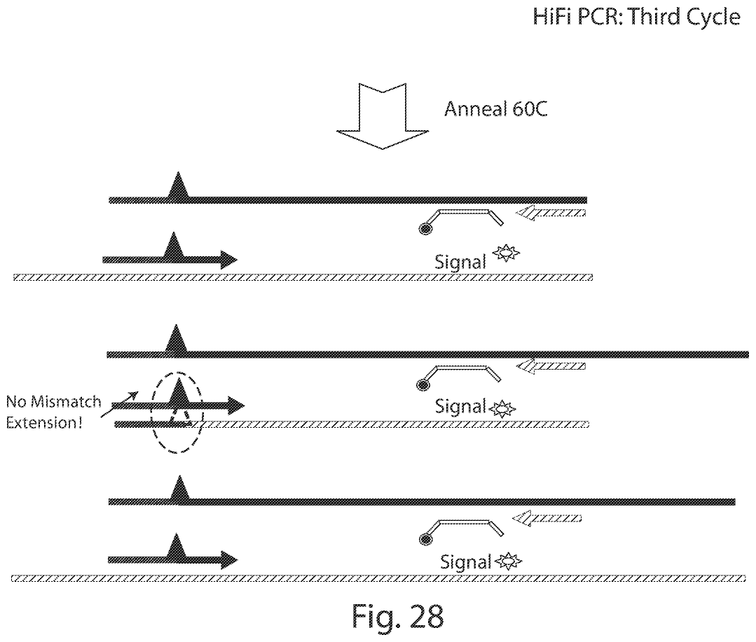

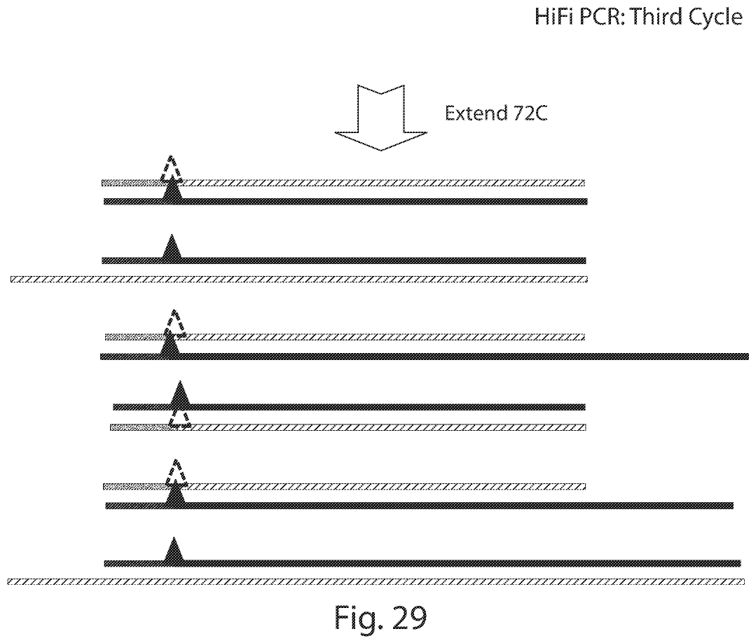

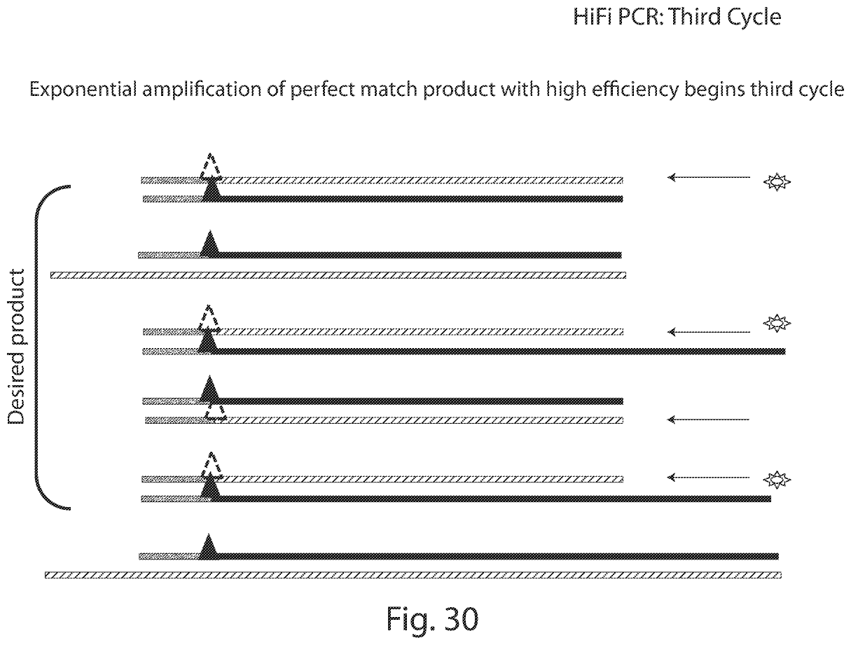

A probe may be useful for methods of the present disclosure, particularly for those methods that include a quantification step. Any one of the methods provided herein can include the use of a probe in the performance of the PCR assay(s), while any one of the compositions of kits provided herein can include one or more probes. Importantly, in some embodiments of any one of the methods provided herein, the probe in one or more or all of the PCR quantification assays is on the same strand as the mismatch primer and not on the opposite strand. It has been found that in so incorporating the probe in a PCR reaction, additional allele specific discrimination can be provided. This is illustrated in FIGS. 11-30.

As an example, a TaqMan® probe is a hydrolysis probe that has a FAM™ or VIC® dye label on the 5′ end, and minor groove binder (MGB) non-fluorescent quencher (NFQ) on the 3′ end. The TaqMan® probe principle generally relies on the 5′-3′ exonuclease activity of Taq® polymerase to cleave the dual-labeled TaqMan® probe during hybridization to a complementary probe-binding region and fluorophore-based detection. TaqMan® probes can increase the specificity of detection in quantitative measurements during the exponential stages of a quantitative PCR reaction.

PCR systems generally rely upon the detection and quantitation of fluorescent dyes or reporters, the signal of which increase in direct proportion to the amount of PCR product in a reaction. For example, in the simplest and most economical format, that reporter can be the double-strand DNA-specific dye SYBR® Green (Molecular Probes). SYBR Green is a dye that binds the minor groove of double stranded DNA. When SYBR Green dye binds to a double stranded DNA, the fluorescence intensity increases. As more double stranded amplicons are produced, SYBR Green dye signal will increase.

In any one of the methods provided herein the PCR may be digital PCR. Digital PCR involves partitioning of diluted amplification products into a plurality of discrete test sites such that most of the discrete test sites comprise either zero or one amplification product. The amplification products are then analyzed to provide a representation of the frequency of the selected genomic regions of interest in a sample. Analysis of one amplification product per discrete test site results in a binary “yes-or-no” result for each discrete test site, allowing the selected genomic regions of interest to be quantified and the relative frequency of the selected genomic regions of interest in relation to one another be determined. In certain aspects, in addition to or as an alternative, multiple analyses may be performed using amplification products corresponding to genomic regions from predetermined regions. Results from the analysis of two or more predetermined regions can be used to quantify and determine the relative frequency of the number of amplification products. Using two or more predetermined regions to determine the frequency in a sample reduces a possibility of bias through, e.g., variations in amplification efficiency, which may not be readily apparent through a single detection assay. Methods for quantifying DNA using digital PCR are known in the art and have been previously described, for example in U.S. patent Publication number US20140242582.

It should be appreciated that the PCR conditions provided herein may be modified or optimized to work in accordance with any one of the methods described herein. Typically, the PCR conditions are based on the enzyme used, the target template, and/or the primers. In some embodiments, one or more components of the PCR reaction is modified or optimized. Non-limiting examples of the components of a PCR reaction that may be optimized include the template DNA, the primers (e.g., forward primers and reverse primers), the deoxynucleotides (dNTPs), the polymerase, the magnesium concentration, the buffer, the probe (e.g., when performing real-time PCR), the buffer, and the reaction volume.

In any of the foregoing embodiments, any DNA polymerase (enzyme that catalyzes polymerization of DNA nucleotides into a DNA strand) may be utilized, including thermostable polymerases. Suitable polymerase enzymes will be known to those skilled in the art, and include E. coli DNA polymerase, Klenow fragment of E. coli DNA polymerase I, T7 DNA polymerase, T4 DNA polymerase, T5 DNA polymerase, Klenow class polymerases, Taq polymerase, Pfu DNA polymerase, Vent polymerase, bacteriophage 29, REDTaq™ Genomic DNA polymerase, or sequenase. Exemplary polymerases include, but are not limited to Bacillus stearothermophilus pol I, Thermus aquaticus (Taq) pol I, Pyrccoccus furiosus (Pfu), Pyrccoccus woesei (Pwo), Thermus flavus (Tfl), Thermus thermophilus (Tth), Thermus litoris (Tli) and Thermotoga maritime (Tma). These enzymes, modified versions of these enzymes, and combination of enzymes, are commercially available from vendors including Roche, Invitrogen, Qiagen, Stratagene, and Applied Biosystems. Representative enzymes include PHUSION® (New England Biolabs, Ipswich, Mass.), Hot MasterTaq™ (Eppendorf), PHUSION® Mpx (Finnzymes), PyroStart® (Fermentas), KOD (EMD Biosciences), Z-Taq (TAKARA), and CS3AC/LA (KlenTaq, University City, Mo.).

Salts and buffers include those familiar to those skilled in the art, including those comprising MgCl2, and Tris-HCl and KCl, respectively. Typically, 1.5-2.0 nM of magnesium is optimal for Taq DNA polymerase, however, the optimal magnesium concentration may depend on template, buffer, DNA and dNTPs as each has the potential to chelate magnesium. If the concentration of magnesium [Mg2+] is too low, a PCR product may not form. If the concentration of magnesium [Mg2+] is too high, undesired PCR products may be seen. In some embodiments the magnesium concentration may be optimized by supplementing magnesium concentration in 0.1 mM or 0.5 mM increments up to about 5 mM.

Buffers used in accordance with the disclosure may contain additives such as surfactants, dimethyl sulfoxide (DMSO), glycerol, bovine serum albumin (BSA) and polyethylene glycol (PEG), as well as others familiar to those skilled in the art. Nucleotides are generally deoxyribonucleoside triphosphates, such as deoxyadenosine triphosphate (dATP), deoxycytidine triphosphate (dCTP), deoxyguanosine triphosphate (dGTP), and deoxythymidine triphosphate (dTTP), which are also added to a reaction adequate amount for amplification of the target nucleic acid. In some embodiments, the concentration of one or more dNTPs (e.g., dATP, dCTP, dGTP, dTTP) is from about 10 μM to about 500 μM which may depend on the length and number of PCR products produced in a PCR reaction.

In some embodiments, the primers used in accordance with the disclosure are modified. The primers may be designed to bind with high specificity to only their intended target (e.g., a particular SNV) and demonstrate high discrimination against further nucleotide sequence differences. The primers may be modified to have a particular calculated melting temperature (Tm), for example a melting temperature ranging from 46° C. to 64° C. To design primers with desired melting temperatures, the length of the primer may be varied and/or the GC content of the primer may be varied. Typically, increasing the GC content and/or the length of the primer will increase the Tm of the primer. Conversely, decreasing the GC content and/or the length of the primer will typically decrease the Tm of the primer. It should be appreciated that the primers may be modified by intentionally incorporating mismatch(es) with respect to the target in order to detect a particular SNV (or other form of sequence non-identity) over another with high sensitivity. Accordingly, the primers may be modified by incorporating one or more mismatches with respect to the specific sequence (e.g., a specific SNV) that they are designed to bind.

In some embodiments, the concentration of primers used in the PCR reaction may be modified or optimized. In some embodiments, the concentration of a primer (e.g., a forward or reverse primer) in a PCR reaction may be, for example, about 0.05 μM to about 1 μM. In particular embodiments, the concentration of each primer is about 1 nM to about 1 μM. It should be appreciated that the primers in accordance with the disclosure may be used at the same or different concentrations in a PCR reaction. For example, the forward primer of a primer pair may be used at a concentration of 0.5 μM and the reverse primer of the primer pair may be used at 0.1 μM. The concentration of the primer may be based on factors including, but not limited to, primer length, GC content, purity, mismatches with the target DNA or likelihood of forming primer dimers.

In some embodiments, the thermal profile of the PCR reaction is modified or optimized. Non-limiting examples of PCR thermal profile modifications include denaturation temperature and duration, annealing temperature and duration and extension time.

The temperature of the PCR reaction solutions may be sequentially cycled between a denaturing state, an annealing state, and an extension state for a predetermined number of cycles. The actual times and temperatures can be enzyme, primer, and target dependent. For any given reaction, denaturing states can range in certain embodiments from about 70° C. to about 100° C. In addition, the annealing temperature and time can influence the specificity and efficiency of primer binding to a particular locus within a target nucleic acid and may be important for particular PCR reactions. For any given reaction, annealing states can range in certain embodiments from about 20° C. to about 75° C. In some embodiments, the annealing state can be from about 46° C. to 64° C. In certain embodiments, the annealing state can be performed at room temperature (e.g., from about 20° C. to about 25° C.).

Extension temperature and time may also impact the allele product yield. For a given enzyme, extension states can range in certain embodiments from about 60° C. to about 75° C.

Quantification of the amounts of the alleles from a quantification assay as provided herein can be performed as provided herein or as otherwise would be apparent to one of ordinary skill in the art. As an example, amplification traces are analyzed for consistency and robust quantification. Internal standards may be used to translate the Cycle threshold to amount of input nucleic acids (e.g., DNA). The amounts of alleles can be computed as the mean of performant assays and can be adjusted for genotype. The wide range of efficient amplifications shows successful detection of low concentration nucleic acids. The amounts provided herein, such as percent donor, in any one of the methods provided can be computed as the trimmed mean of all performant assays (e.g., nanograms non-native allele to nanograms native allele ratio). In some embodiments, the amounts as provided herein, such as the percent donor, in any one of the methods provided can be computed as the median of all performant assays. Amounts can be determined with an adjustment for genotypes.

It has been found that the methods and compositions provided herein can be used to detect low-level nucleic acids, such as non-native nucleic acids, in a sample. Accordingly, the methods provided herein can be used on samples where detection of relatively rare nucleic acids is needed. In some embodiments, any one of the methods provided herein can be used on a sample to detect non-native nucleic acids that are less than 1.5% of the nucleic acids in the sample. In other embodiments, any one of the methods provided herein can be used on a sample where less than 1.3%, 1.2%, 1.1%, 1%, 0.9%, 0.8%, 0.7%, 0.6%, 0.5% 0.3%, 0.2%, 0.1%, 0.09%, 0.05%, 0.03%, or 0.01% of the nucleic acids in the sample are non-native. In other embodiments, any one of the methods provided herein can be used on a sample where at least 0.005%, 0.01%, 0.03% or 0.05% of the nucleic acids are non-native. In still other embodiments of any one of the methods provided herein, at least 0.005% but less than 1.3%, 1.2%, 1.1%, 1%, 0.9%, 0.8%, 0.7%, 0.6%, 0.5% 0.3%, 0.2%, 0.1%, 0.09%, 0.05%, 0.03%, or 0.01% of the nucleic acids in the sample are non-native.

Because of the ability to determine amounts of non-native nucleic acids, even at low levels, the methods and compositions provided herein can be used to assess a risk in a subject, such as a transplant recipient. A “risk” as provided herein, refers to the presence or absence of any undesirable condition in a subject (such as a transplant recipient), or an increased likelihood of the presence or absence of such a condition, e.g., transplant rejection. As provided herein “increased risk” refers to the presence of any undesirable condition in a subject or an increased likelihood of the presence of such a condition. As provided herein, “decreased risk” refers to the absence of any undesirable condition in a subject or a decreased likelihood of the presence (or increased likelihood of the absence) of such a condition.

As an example, early detection of rejection following implantation of a transplant (e.g., a heart transplant) can facilitate treatment and improve clinical outcomes. Transplant rejection remains a major cause of graft failure and late mortality and generally requires lifelong surveillance monitoring. Treatment of transplant rejections with immunosuppressive therapy has been shown to improve treatment outcomes, particularly if rejection is detected early. Transplant rejection is typically monitored using a catheter-based endomyocardial biopsy (EMB). This invasive procedure, however, is associated with risks and discomfort for a patient, and may be particularly disadvantageous for pediatric patients. Accordingly, provided herein are sensitive, specific, cost effective, and non-invasive techniques for the surveillance of subjects, such as transplant recipients. Such techniques have been found to allow for the detection of transplant rejection at an early stage. Such techniques can also be used to monitor organ recovery and in the selection and monitoring of a treatment or therapy, such as an anti-rejection treatment or anti-infection treatment, thus improving a patient's recovery and increasing survival rates. In some embodiments of any one of the methods provided herein, the method can be performed on one or more samples from the subject as early as within 14 or 24 hours of surgery, such as transplant surgery. In some embodiments of any one of the methods provided herein, the method can be performed on one or more samples from the subject as early as within 14 or 24 hours of cross-clamp removal, such as in a heart transplant. In any one of the methods provided herein, an amount of the non-native nucleic acids in a subject can be obtained for one or more samples taken within 14 or 24 hours of surgery, such as transplant surgery. In any one of the methods provided herein, an amount of the non-native nucleic acids in a subject can be obtained for one or more samples taken within 14 or 24 hours of cross-clamp removal, such as in a heart transplant. A clinician can then make an assessment of the subject with this amount.

Accordingly, in some embodiments of any one of the methods provided, the subject is a recipient of a transplant, and the risk is a risk associated with the transplant. In some embodiments of any one of the methods provided, the risk associated with the transplant is risk of transplant rejection, an anatomical problem with the transplant or injury to the transplant. In some embodiments of any one of the methods provided, the injury to the transplant is initial or ongoing injury. In some embodiments of any one of the methods provided, the risk associated with the transplant is an acute condition or a chronic condition. In some embodiments of any one of the methods provided, the acute condition is transplant rejection including cellular rejection or antibody mediated rejection. In some embodiments of any one of the methods provided, the chronic condition is graft vasculopathy. In some embodiments of any one of the methods provided, the risk associated with the transplant is indicative of the severity of the injury. In some embodiments of any one of the methods provided, the risk associated with the transplant is risk or status of an infection.

As used herein, “transplant” refers to the moving of an organ from a donor to a recipient for the purpose of replacing the recipient's damaged or absent organ. The transplant may be of one organ or more than one organ. In some embodiments, the term “transplant” refers to a transplanted organ or organs, and such meaning will be clear from the context the term is used. Examples of organs that can be transplanted include, but are not limited to, the heart, kidney(s), kidney, liver, lung(s), pancreas, intestine, etc. Any one of the methods provided herein may be used on a sample from a subject that has undergone a transplant of any one or more of the organs provided herein. In some embodiments, the transplant is a heart transplant.

The risk in a recipient of a transplant can be determined, for example, by assessing the amount of non-native cf-DNA, such as donor-specific cell-free-DNA (DS cf-DNA), a biomarker for cellular injury related to transplant rejection. DS cf-DNA refers to DNA that presumably is shed from the transplanted organ, the sequence of which matches (in whole or in part) the genotype of the donor who donated the transplanted organ.

The risk in a recipient of a transplant can be determined, for example, by assessing the amount of non-native cf-DNA, such as donor-specific cell-free DNA, as described herein using any one of the methods provided.

In some embodiments, any one of the methods provided herein can comprise correlating an increase in non-native nucleic acids and/or an increase in the ratio, or percentage, of non-native nucleic acids relative to native or total nucleic acids, with an increased risk of a condition, such as transplant rejection. In some embodiments of any one of the methods provided herein, correlating comprises comparing a level (e.g., concentration, ratio or percentage) of non-native nucleic acids to a threshold value to identify a subject at increased or decreased risk of a condition. In some embodiments of any one of the methods provided herein, a subject having an increased amount of non-native nucleic acids compared to a threshold value is identified as being at increased risk of a condition. In some embodiments of any one of the methods provided herein, a subject having a decreased or similar amount of non-native nucleic acids compared to a threshold value is identified as being at decreased risk of a condition.

As used herein, “amount” refers to any quantitative value for the measurement of nucleic acids and can be given in an absolute or relative amount. Further, the amount can be a total amount, frequency, ratio, percentage, etc. As used herein, the term “level” can be used instead of “amount” but is intended to refer to the same types of values.

“Threshold” or “threshold value”, as used herein, refers to any predetermined level or range of levels that is indicative of the presence or absence of a condition or the presence or absence of a risk. The threshold value can take a variety of forms. It can be single cut-off value, such as a median or mean. It can be established based upon comparative groups, such as where the risk in one defined group is double the risk in another defined group. It can be a range, for example, where the tested population is divided equally (or unequally) into groups, such as a low-risk group, a medium-risk group and a high-risk group, or into quadrants, the lowest quadrant being subjects with the lowest risk and the highest quadrant being subjects with the highest risk. The threshold value can depend upon the particular population selected. For example, an apparently healthy population will have a different ‘normal’ range. As another example, a threshold value can be determined from baseline values before the presence of a condition or risk or after a course of treatment. Such a baseline can be indicative of a normal or other state in the subject not correlated with the risk or condition that is being tested for. In some embodiments, the threshold value can be a baseline value or value from another point in time, such as a prior point in time, of the subject being tested. Accordingly, the predetermined values selected may take into account the category in which the subject falls. Appropriate ranges and categories can be selected with no more than routine experimentation by those of ordinary skill in the art.

Changes in the levels of non-native nucleic acids can also be monitored over time. For example, a change from a threshold value in the amount, such as ratio or percentage, of non-native nucleic acids can be used as a non-invasive clinical indicator of risk, e.g., risk associated with transplant. This can allow for the measurement of variations in a clinical state and/or permit calculation of normal values or baseline levels. In organ transplantation, this can form the basis of an individualized non-invasive screening test for rejection or a risk of a condition associated thereto. Generally, as provided herein, the amount, such as the ratio or percent, of non-native nucleic acids can be indicative of the presence or absence of a risk associated with a condition, such as risk associated with a transplant, such as rejection, in the recipient, or can be indicative of the need for further testing or surveillance. In one embodiment of any one of the methods provided herein, the method may further include an additional test(s) for assessing a condition, such as transplant rejection, transplant injury, etc. The additional test(s) may be any one of the methods provided herein.

In some embodiments of any one of the methods provided herein in regard to a heart transplant recipient, such threshold is equal to or greater than 0.8%, 0.9%, or 1%, wherein a level above, respectively, is indicative of an increased risk and wherein a level at or below is indicative of a decreased risk. In some embodiments of any one of the methods provided herein in regard to a heart transplant recipient, such threshold is equal greater than 1.1%, 1.2% or 1.3%, wherein a level above is indicative of an increased risk and wherein a level at or below is indicative of a decreased risk.

In some embodiments of any one of the methods provided herein, where a non-native nucleic acid amount, such as ratio or percentage, is determined to be above a threshold value, any one of the methods provided herein can further comprise performing another test on the subject or sample therefrom. Such other tests can be any other test known by one of ordinary skill in the art to be useful in determining the presence or absence of a risk, e.g., in a transplant recipient. In some embodiments, the other test is any one of the methods provided herein. In some embodiments of any one of the methods provided herein, the subject is a transplant recipient and the other test is a determination of the level of BNP and/or troponin in the transplant recipient. In other embodiments of any one of the methods provided herein, the other test in addition to the level of BNP and/or troponin or in place thereof is an echocardiogram.

In some embodiments of any one of the methods provided herein, where the non-native nucleic acid amount, such as the ratio or percentage, is determined to be less than a threshold value no further testing may be needed or recommended to the subject and/or no treatment is needed or suggested to the subject. While in some embodiments of any one of the methods provided herein, it may be determined such subjects may still need monitoring over time. It should be appreciated that other thresholds may be utilized as embodiments of the invention. In some embodiments of any one of the methods provided herein, the method may further comprise further testing or recommending further testing to the subject and/or treating or suggesting treatment to the subject. In some of these embodiments, the further testing is any one of the methods provided herein.

In some embodiments of any one of the methods provided herein, the method may further comprise determining a treatment regimen based on the amount(s). “Determining a treatment regimen”, as used herein, refers to the determination of a course of action for the treatment of the subject. In one embodiment of any one of the methods provided herein, determining a treatment regimen includes determining an appropriate therapy or information regarding an appropriate therapy to provide to a subject. In some embodiments of any one of the methods provided herein, the determining includes providing an appropriate therapy or information regarding an appropriate therapy to a subject. As used herein, information regarding a treatment or therapy or monitoring may be provided in written form or electronic form. In some embodiments, the information may be provided as computer-readable instructions. In some embodiments, the information may be provided orally.

In some of these embodiments, the treating is an anti-rejection treatment or anti-infection. In some embodiments, the information is provided in written form or electronic form. In some embodiments, the information may be provided as computer-readable instructions.

Anti-rejection therapies include, for example, the administration of an immunosuppressive to a transplant recipient. “Administering” or “administration” or “administer” or the like means providing a material to a subject in a manner that is pharmacologically useful directly or indirectly. Thus, the term includes directing, such as prescribing, the subject or another party to administer the material. Administration of a treatment or therapy may be accomplished by any method known in the art (see, e.g., Harrison's Principle of Internal Medicine, McGraw Hill Inc.). Preferably, administration of a treatment or therapy occurs in a therapeutically effective amount. Compositions for different routes of administration are known in the art (see, e.g., Remington's Pharmaceutical Sciences by E. W. Martin).

Immunosuppressives include, but are not limited to, corticosteroids (e.g., prednisolone or hydrocortisone), glucocorticoids, cytostatics, alkylating agents (e.g., nitrogen mustards (cyclophosphamide), nitrosoureas, platinum compounds, cyclophosphamide (Cytoxan)), antimetabolites (e.g., folic acid analogues, such as methotrexate, purine analogues, such as azathioprine and mercaptopurine, pyrimidine analogues, and protein synthesis inhibitors), cytotoxic antibiotics (e.g., dactinomycin, anthracyclines, mitomycin C, bleomycin, mithramycin), antibodies (e.g., anti-CD20, anti-IL-1, anti-IL-2Ralpha, anti-T-cell or anti-CD-3 monoclonals and polyclonals, such as Atgam, and Thymoglobuline), drugs acting on immunophilins, ciclosporin, tacrolimus, sirolimus, interferons, opiods, TNF-binding proteins, mycophenolate, fingolimod and myriocin. In some embodiments, anti-rejection therapy comprises blood transfer or marrow transplant. Therapies can also include therapies for treating systemic conditions, such as sepsis. The therapy for sepsis can include intravenous fluids, antibiotics, surgical drainage, early goal directed therapy (EGDT), vasopressors, steroids, activated protein C, drotrecogin alfa (activated), oxygen and appropriate support for organ dysfunction. This may include hemodialysis in kidney failure, mechanical ventilation in pulmonary dysfunction, transfusion of blood products, and drug and fluid therapy for circulatory failure. Ensuring adequate nutrition—preferably by enteral feeding, but if necessary by parenteral nutrition—can also be included particularly during prolonged illness. Other associated therapies can include insulin and medication to prevent deep vein thrombosis and gastric ulcers.

In some embodiments, wherein infection is indicated, therapies for treating a recipient of a transplant can also include therapies for treating a bacterial, fungal and/or viral infection. Such therapies include antibiotics. Other examples include, but are not limited to, amebicides, aminoglycosides, anthelmintics, antifungals, azole antifungals, echinocandins, polyenes, diarylquinolines, hydrazide derivatives, nicotinic acid derivatives, rifamycin derivatives, streptomyces derivatives, antiviral agents, chemokine receptor antagonist, integrase strand transfer inhibitor, neuraminidase inhibitors, NNRTIs, NSSA inhibitors, nucleoside reverse transcriptase inhibitors (NRTIs), protease inhibitors, purine nucleosides, carbapenems, cephalosporins, glycylcyclines, leprostatics, lincomycin derivatives, macrolide derivatives, ketolides, macrolides, oxazolidinone antibiotics, penicillins, beta-lactamase inhibitors, quinolones, sulfonamides, and tetracyclines. Other such therapies are known to those of ordinary skill in the art. Any one of the methods provided herein can include administering or suggesting an anti-infection treatment to the subject (including providing information about the treatment to the subject, in some embodiments). In some embodiments, an anti-infection treatment may be a reduction in the amount or frequency in an immunosuppressive therapy or a change in the immunosuppressive therapy that is administered to the subject. Other therapies are known to those of ordinary skill in the art.

It has been found that particularly useful to a clinician is a report that contains the amount(s), result(s) or other value(s) provided herein. In one aspect, therefore such reports are provided. Reports may be in oral, written (or hard copy) or electronic form, such as in a form that can be visualized or displayed. In some embodiments, the “raw” results for each assay as provided herein are provided in a report, and from this report, further steps can be taken to analyze the amount(s) of non-native nucleic acids (such as donor-specific cell-free DNA). In other embodiments, the report provides multiple values for the amounts non-native nucleic acids (such as donor-specific cell-free DNA) for a subject. From the amounts, in some embodiments, a clinician may assess the need for a treatment for the subject or the need to monitor the subject over time.

Any one of the methods provided herein can comprise extracting nucleic acids, such as cell-free DNA, from a sample obtained from a subject, such as a recipient of a transplant. Such extraction can be done using any method known in the art or as otherwise provided herein (see, e.g., Current Protocols in Molecular Biology, latest edition, or the QlAamp circulating nucleic acid kit or other appropriate commercially available kits). An exemplary method for isolating cell-free DNA from blood is described. Blood containing an anti-coagulant such as EDTA or DTA is collected from a subject. The plasma, which contains cf-DNA, is separated from cells present in the blood (e.g., by centrifugation or filtering). An optional secondary separation may be performed to remove any remaining cells from the plasma (e.g., a second centrifugation or filtering step). The cf-DNA can then be extracted using any method known in the art, e.g., using a commercial kit such as those produced by Qiagen. Other exemplary methods for extracting cf-DNA are also known in the art (see, e.g., Cell-Free Plasma DNA as a Predictor of Outcome in Severe Sepsis and Septic Shock. Clin. Chem. 2008, v. 54, p. 1000-1007; Prediction of MYCN Amplification in Neuroblastoma Using Serum DNA and Real-Time Quantitative Polymerase Chain Reaction. JCO 2005, v. 23, p. 5205-5210; Circulating Nucleic Acids in Blood of Healthy Male and Female Donors. Clin. Chem. 2005, v. 51, p. 131′7-1319; Use of Magnetic Beads for Plasma Cell-free DNA Extraction: Toward Automation of Plasma DNA Analysis for Molecular Diagnostics. Clin. Chem. 2003, v. 49, p. 1953-1955; Chiu R W K, Poon L L M, Lau T K, Leung T N, Wong E M C, Lo Y M D. Effects of blood-processing protocols on fetal and total DNA quantification in maternal plasma. Clin Chem 2001; 47:1607-1613; and Swinkels et al. Effects of Blood-Processing Protocols on Cell-free DNA Quantification in Plasma. Clinical Chemistry, 2003, vol. 49, no. 3, 525-526).

As used herein, the sample from a subject can be a biological sample. Examples of such biological samples include whole blood, plasma, serum, etc. In some embodiments of any one of the methods provided herein, addition of further nucleic acids, e.g., a standard, to the sample can be performed.

In some embodiments of any one of the methods provided herein, an early additional amplification step is performed. An exemplary method of amplification is as follows, and such a method can be included in any one of the methods provided herein. ˜15 ng of cell free plasma DNA is amplified in a PCR using Q5 DNA polymerase with approximately ˜100 targets where pooled primers were at 6 uM total. Samples undergo approximately 35 cycles. Reactions are in 25 ul total. After amplification, samples can be cleaned up using several approaches including AMPURE bead cleanup, bead purification, or simply Exosap it, or Zymo. Such an amplification step was used in some methods as provided herein.

The present disclosure also provides methods for determining a plurality of SNV targets for use in any one of the methods provided herein or from which any one of the compositions of primers can be derived. A method of determining a plurality of SNV targets, in some embodiments comprises a) identifying a plurality of highly heterozygous SNVs in a population of individuals, b) designing one or more primers spanning each SNV, c) selecting sufficiently specific primers, d) evaluating multiplexing capabilities of primers, such as at a common melting temperature and/or in a common solution, and e) identifying sequences that are evenly amplified with the primers or a subset thereof.

As used herein, “highly heterozygous SNVs” are those with a minor allele at a sufficiently high percentage in a population. In some embodiments, the minor allele is at least 25%, 26%, 27%, 28%, 29%, 30%, 31%, 32%, 33%, 34% or 35% or more in the population. In any one of these embodiments, the minor allele is less than 50%, 49%, 45% or 40% in the population. Such SNVs increase the likelihood of providing a target that is different between the native and non-native nucleic acids.

Primers were designed to generally span a 70 bp window but some other window may also be selected, such as one between 60 bps and 80 bps. Also, generally, it was desired for the SNV to fall about in the middle of this window. For example, for a 70 bp window, the SNV was between bases 20-50, such as between bases 30-40. The primers as provided herein were designed to be adjacent to the SNV.

As used herein, “sufficiently specific primers”, were those that demonstrated discrimination between amplification of the intended allele versus amplification of the unintended allele. Thus, with PCR a cycle gap was desired between amplification of the two. In one embodiment, the cycle gap was at least a 5, 6, 7 or 8 cycle gap.

Further, sequences were selected based on melting temperatures, generally those with a melting temperature of between 45-55 degrees C. were selected as “moderate range sequences”. Other temperature ranges may be desired and can be determined by one of ordinary skill in the art. A “moderate range sequence” generally is one that can be amplified in a multiplex amplification format within the temperature. In some embodiments, the gc % content was between 30-70%, such as between 33-66%.

In one embodiment of any one of the methods provided herein, the method can further comprise excluding sequences associated with difficult regions. “Difficult regions” are any regions with content or features that make it difficult to reliably make predictions about a target sequence or are thought to not be suitable for multiplex amplification. Such regions include syndromic regions, low complexity regions, regions with high GC content or that have sequential tandem repeats. Other such features can be determined or are otherwise known to those of ordinary skill in the art.

In some embodiments of any one of the methods provided herein, the primer pairs are designed to be compatible for use in a quantitative assay as provided herein. For example, the primer pairs can be designed to prevent primer dimers and/or limit the number of off-target binding sites. It should be appreciated that the plurality of primer pairs of any one of the methods, compositions or kits provided may be optimized or designed in accordance with any one of the methods described herein.

Various aspects of the present invention may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Also, embodiments of the invention may be implemented as one or more methods, of which an example has been provided. The acts performed as part of the method(s) may be ordered in any suitable way. Accordingly, embodiments may be constructed in which acts are performed in an order different from illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The following description provides examples of the methods provided herein.

### EXAMPLES

### Example 1—With Recipient and Donor Genotype Information

SNV Target Selection

Identification of targets for multiplexing in accordance with the disclosure may include one or more of the following steps, as presently described. First, highly heterozygous SNPs can be screened on several ethnic control populations (Hardy-Weinberg p>0.25), excluding known difficult regions. Difficult regions include syndromic regions likely to be abnormal in patients and regions of low complexity, including centromeres and telomeres of chromosomes. Target fragments of desired lengths can then be designed in silico. Specifically, two 20-26 bp primers spanning each SNP's 70 bp window can be designed. All candidate primers can then be queried to GCRh37 using BLAST. Those primers that were found to be sufficiently specific can be retained, and monitored for off-target hits, particularly at the 3′ end of the fragment. The off-target candidate hits can be analyzed for pairwise fragment generation that would survive size selection. Selected primers can then be subjected to an in silico multiplexing evaluation. The primers' computed melting temperatures and guanine-cytosine percentages (GC %) can be used to filter for moderate range sequences. An iterated genetic algorithm and simulated annealing can be used to select candidate primers compatible for 400 targets, ultimately resulting in the selection of 800 primers. The 800 primers can be generated and physically tested for multiplex capabilities at a common melting temperature in a common solution. Specifically, primers can be filtered based on even amplification in the multiplex screen and moderate read depth window. Forty-eight assays can be designed for MOMA using the top performing multiplexed SNPs. Each SNP can have a probe designed in WT/MUT at four mismatch choices; eight probes per assay. The new nested primers can be designed within the 70 bp enriched fragments. Finally, the primers can be experimentally amplified to evaluate amplification efficiency (8 probes×48 assays in triplicate, using TAQMAN™).

A Priori Genotyping Informativeness of Each Assay

Using, for example, known or possible native and non-native genotypes at each assayed SNP, a subset of informative assays was selected. Note that subject homozygous sites can be used where the non-native is any other genotype. Additionally, if the non-native genotype is not known, it can be inferred. Genotypes may also be learned through sequencing, SNP microarray, or application of a MOMA assay on known 0% (clean recipient) samples.

Post Processing Analysis of Multiplex Assay Performance

Patient-specific MOMA probe biases can be estimated across an experimental cohort. Selection iteratively can be refined to make the final non-native percent call.

Reconstruction Experiment

The sensitivity and precision of the assay can be evaluated using reconstructed plasma samples with known mixing ratios. Specifically, the ratios of 1:10, 1:20, 1:100, 1:200, and 1:1000 can be evaluated. Generally, primers for 95 SNV targets can be used as described herein in some embodiments.

To work without non-native genotype information, the following procedure may be performed to infer informative assays and allow for quantification of non-native-specific cell-free DNA in plasma samples. All assays can be evaluated for performance in the full information scenario. This procedure thus assumed clean AA/AB/BB genotypes at each assay and unbiased behavior of each quantification. With native genotype, assays known to be homozygous in the subject can be selected. Contamination can be attributed to the non-native nucleic acids, and the assay collection created a tri-modal distribution with three clusters of assays corresponding to the non-, half, and fully-informative assays. With sufficient numbers of recipient homozygous assays, the presence of non-native fully informative assays can be assumed.

If the native genotype is homozygous and known, then if a measurement that is not the non-native genotype is observed, the probes which are truly non-native-homozygous will have the highest cluster and equal the guess whereas those that are non-native heterozygous will be at half the guess. A probability distribution can be plotted and an expectation maximization algorithm (EM) can be employed to infer non-native genotype. Such can be used to infer the non-native genotype frequency in any one of the methods provided herein.

Accordingly, an EM algorithm was used to infer the most likely non-native genotypes at all assayed SNV targets. With inferred non-native genotypes, quantification may proceed as in the full-information scenario. EM can begin with the assumption that the minor allele ratio found at an assay follows a tri-modal distribution, one for each combination of subject and non-native, given all assays are “AA” in the subject (or flipped from “BB” without loss of generality). With all non-native genotypes unknown, it is possible to bootstrap from the knowledge that any assays exhibiting nearly zero minor allele are non-native AA, and the highest is non-native BB. Initial guesses for all non-native genotypes were recorded, and the mean of each cluster calculated. Enforcing that the non-native BB assays' mean is twice that of the non-native AB restricts the search. The algorithm then reassigns guessed non-native genotypes based on the clusters and built-in assumptions. The process was iterative until no more changes were made. The final result is a set of the most likely non-native genotypes given their measured divergence from the background. Generally, every target falls into the model; a result may be tossed if between groups after maximization.

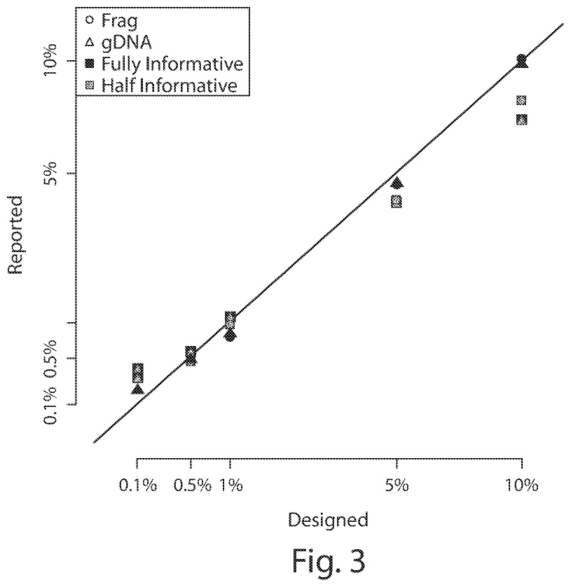

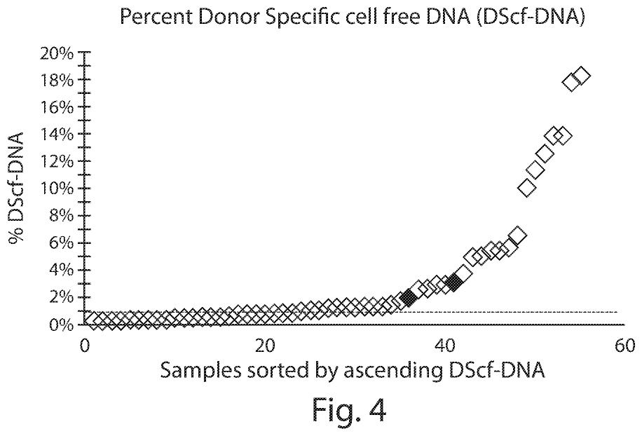

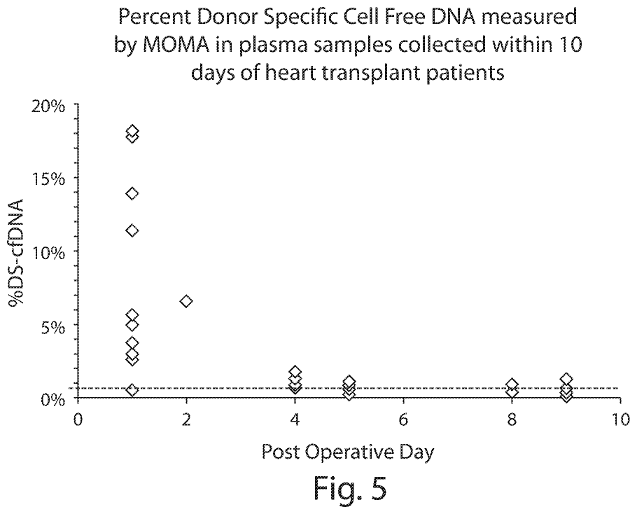

Results of the reconstruction experiment demonstrate proof of concept (FIG. 3). One target is fully informative where there is a homozygous donor against a homozygous recipient (shaded data points). The other target is half informative where there is a heterozygous donor against a homozygous recipient (open data points). In addition, plasma samples from transplant recipient patients were analyzed with a mismatch method (FIG. 4). All data comes from patients who have had biopsies. Dark points denote rejection. Further data shown in FIG. 5, demonstrate that a mismatch method as provided herein worked with real plasma samples. After transplant surgery, the donor percent levels dropped off. Generally, primers for 95 SNV targets as described herein were used.

### Example 2—with Recipient but not Donor Genotype Information

To work without donor genotype information, the following procedure may be performed to infer informative assays and allow for quantification of donor-specific cell-free DNA in plasma samples. All assays were evaluated for performance in the full information scenario. This procedure thus assumed clean AA/AB/BB genotypes at each assay and unbiased behavior of each quantification. With recipient genotype, assays known to be homozygous in the recipient were selected. Any contamination was attributed to the donor nucleic acids, and the assay collection created a tri-modal distribution with three clusters of assays corresponding to the non-, half, and fully-informative assays. With sufficient numbers of recipient homozygous assays the presence of donor fully informative assays can be assumed.

If recipient genotype is homozygous and known, then if a measurement that is not the recipient genotype is observed, the probes which are truly donor homozygous will have the highest cluster and equal the guess whereas those that are donor heterozygous will be at half the guess. A probability distribution can be plotted and an expectation maximization algorithm (EM) can be employed to infer donor genotype. Such can be used to infer the donor genotype frequency in any one of the methods provided herein. Accordingly, an EM algorithm was used to infer the most likely donor genotypes at all assayed SNV targets. With inferred donor genotypes, quantification may proceed as in the full-information scenario. EM can begin with the assumption that the minor allele ratio found at an assay follows a tri-modal distribution, one for each combination of recipient and donor, given all assays are “AA” in the recipient (or flipped from “BB” without loss of generality). With all donor genotypes unknown, it is possible to bootstrap from the knowledge that any assays exhibiting nearly zero minor allele are donor AA, and the highest is donor BB. Initial guesses for all donor genotypes were recorded, and the mean of each cluster calculated. Enforcing that the donor BB assays' mean is twice that of the donor AB restricts the search. The algorithm then reassigns guessed donor genotypes based on the clusters and built-in assumptions. The process was iterative until no more changes were made. The final result is a set of the most likely donor genotypes given their measured divergence from the background. Generally, every target falls into the model; a result may be tossed if between groups after maximization.

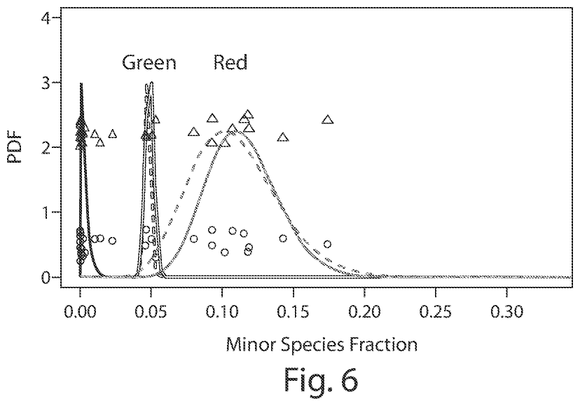

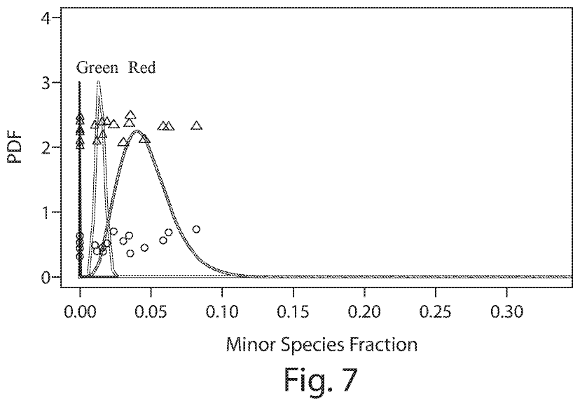

FIG. 6 shows exemplary results from plasma samples handled in this manner. The x-axis is the donor % for any assay found recipient homozygous. The rows of points represent individual PCR assay results. The bottom-most row of circles represents the initial guess of donor genotypes, some AA, some A/B and some BB. Then the solid curves were drawn representing Beta distributions centered on the initial assays, red for homozygous (fully informative) and green for heterozygous (half informative) with black curves representing the distribution of non-informative assays or background noise. The assays were re-assigned updated guesses in the second row. Second row's curves use dashed lines. The top row is the final estimate because no change occurred. Double the peak of the green dashed curve corresponds to the maximum likelihood donor % call, at around 10%, or equal to the mean of the red curve.

A reconstruction experiment (Recon1) using DNA from two individuals were created at 10%, 5%, 1%, 0.5%, and 0.1%. All mixes were amplified with a multiplex library of targets, cleaned, then quantitatively genotyped using a MOMA method. The analysis was performed with genotyping each individual in order to know their true genotypes. Informative targets were determined using prior knowledge of the genotype of the major individual (looking for homozygous sites), and where the second individual was different, and used to calculate fractions (percentage) using informative targets. The fractions were then calculated (depicted in black to denote With Genotype information).

A second reconstruction experiment (Recon2), beginning with two individuals, major and minor were also created at 10%, 5%, 1%, 0.5%, and 0.1%. All mixes were amplified with the multiplex library of targets, cleaned, then quantitatively genotyped using a MOMA method. The analysis was performed with genotyping each individual in order to know their true genotypes. Informative targets were determined using prior knowledge of the genotype of the second individual as described above. The fractions were then calculated (depicted in black to denote With Genotype information).

These reconstructions were run again the next day (Recon3).

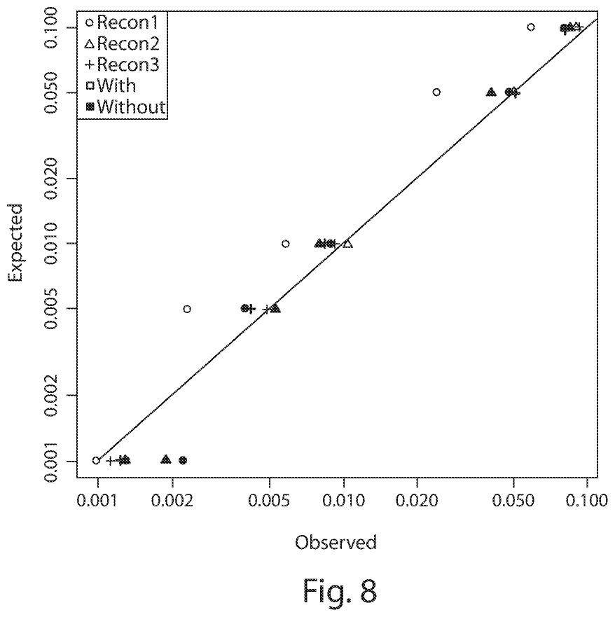

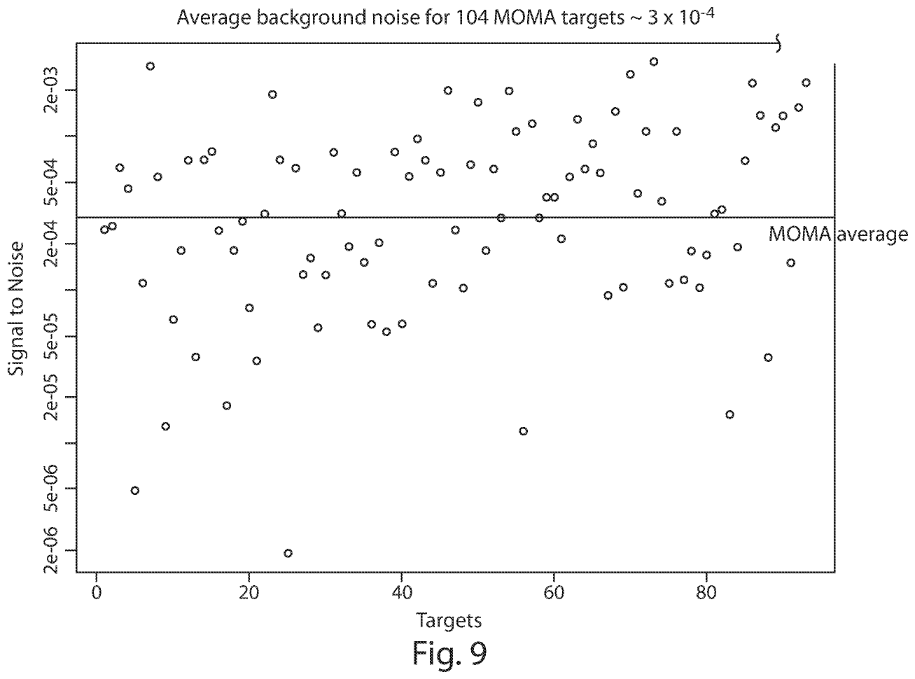

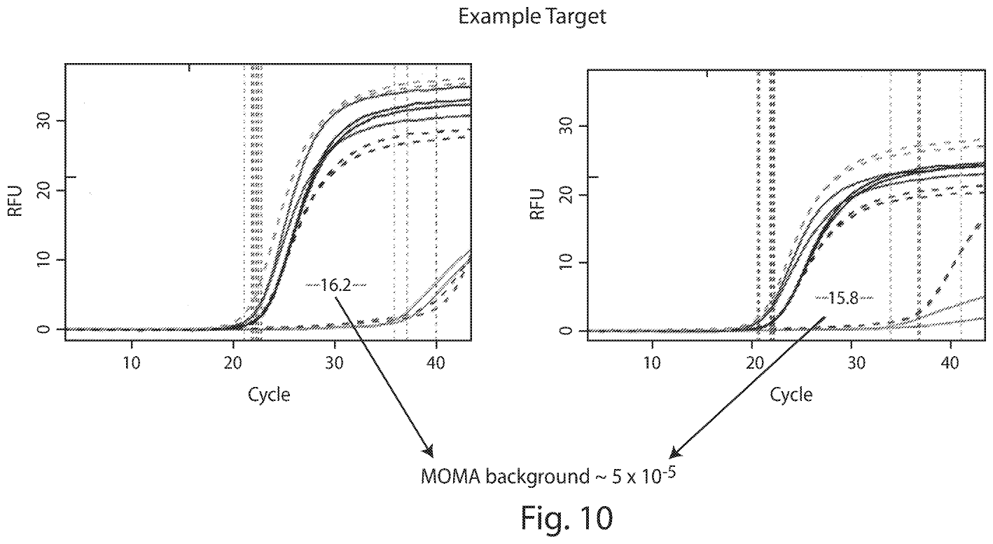

The same reconstruction samples (Recon 1, 2, 3) were then analyzed again without using genotyping information from the second individual (minor DNA contributor) but only genotyping information available for the first individual (major DNA contributor). Approximately 38-40 targets were used to calculate fractions without genotyping (simulating without donor) shaded (FIG. 8). It was found that each target that was recipient homozyous was possibly useful. The circles were the first guess, just thresholding, those on the right were thought to be fully informative and those on the left not. The triangles along the top were the same targets, but for the final informativity decisions they were recolored. It was found the expectation maximization was superior to simple thresholding.

### Example 3—Reconstruction Experiments with Trimmed Mean, Median and Untrimmed Mean

A reconstruction experiment was performed, wherein two samples of DNA were mixed at varying proportions to test the accuracy and precision of MOMA assays. The results are presented below with three types of output measure, the trimmed mean, the median, and the untrimmed means.

| Samples | Trimmed | | Raw | Intended | Useful |
|---|---|---|---|---|---|
| of Run | Mean | Median | Mean | Percentage | Targets |
| Tube1 | 101.90% | 99.97% | 102.53% | 100.00% | 21 |
| Tube2 | 9.66% | 10.03% | 9.77% | 10.00% | 21 |
| Tube3 | 4.83% | 4.81% | 5.00% | 5.00% | 21 |
| Tube4 | 0.96% | 0.95% | 0.96% | 1.00% | 21 |
| Tube5 | 0.58% | 0.55% | 0.67% | 0.50% | 29 |
| Tube6 | 0.16% | 0.10% | 1.02% | 0.10% | 19 |
| Tube7 | 0.09% | 0.02% | 0.92% | 0.00% | 18 |
| Tube8 | NaN | NA | NaN | None | 0 |
| Tube9 | 2.05% | 1.91% | 2.20% | 2.00% | 25 |
| Tube10 | 1.86% | 1.71% | 2.11% | 1.75% | 30 |
| Tube11 | 1.41% | 1.44% | 1.44% | 1.50% | 29 |
| Tube12 | 1.21% | 1.23% | 1.26% | 1.25% | 30 |
| Tube13 | 0.79% | 0.81% | 0.84% | 0.75% | 27 |
| Tube14 | 0.27% | 0.25% | 0.29% | 0.25% | 29 |

Tube 8 had no DNA, the negative control sample accurately reflects a lack of useful targets and “NA” for the donor %. The trimmed mean drops two of the lowest reporting targets and two of the highest, reducing the impact of outliers. The median reports the center-most value. The raw mean is the mean as standardly defined. The final column is the number of targets used in the analysis, after paring down from the 94 candidate targets to just those informative genotypes with this particular recipient/donor pair, and also filtering misbehaving targets or poorly amplified targets which would yield unreliable values.

It was found that the raw mean is strongly biased by individual outlier target values. The median was closer in absolute value to the “intended percentage” than the other two candidate measures in seven of thirteen samples. The raw mean was closest in five, and the trimmed was closest in three. Overall the median was more accurate more often.

Another reconstruction experiment was performed as described above.

| Samples of | Trimmed | | Raw | Intended | Useful |
|---|---|---|---|---|---|
| Run | Mean | Median | Mean | Percentage | Targets |
| Tube1 | 99.19% | 99.89% | 98.68% | 100.00% | 20 |
| Tube2 | 8.61% | 8.50% | 13.71% | 10.00% | 19 |
| Tube3 | NaN | NA | NaN | 5.00% | 0 |
| Tube4 | 1.47% | 0.92% | 8.48% | 1.00% | 17 |
| Tube5 | 1.04% | 0.50% | 5.88% | 0.50% | 22 |
| Tube6 | 0.09% | 0.08% | 0.11% | 0.10% | 23 |
| Tube7 | 0.03% | 0.02% | 0.05% | 0.00% | 24 |
| Tube8 | NaN | NA | NaN | None | 0 |
| Tube9 | 1.68% | 1.69% | 1.79% | 2.00% | 24 |
| Tube10 | 1.32% | 1.23% | 1.43% | 1.75% | 25 |
| Tube11 | 1.28% | 1.21% | 1.29% | 1.50% | 24 |
| Tube12 | 1.19% | 1.21% | 1.20% | 1.25% | 23 |
| Tube13 | 0.65% | 0.60% | 0.68% | 0.75% | 25 |
| Tube14 | 0.25% | 0.23% | 0.28% | 0.25% | 22 |
| Tube15 | 5.88% | 5.60% | 5.87% | 7.14% | 25 |

Tube 3 was an unintended sample failure, believed to be due to poor library amplification. Again, the raw mean is strongly biased by individual outlier target values. The median was again closer in absolute value to the “intended percentage” than the other two candidate measures in five of thirteen samples. The raw mean was closest in five, and the trimmed was closest in four.

Another reconstruction experiment was performed as described above.

| Samples | Trimmed | | Raw | Intended | Useful |
|---|---|---|---|---|---|
| of Run | Mean | Median | Mean | Percentage | Targets |
| Tube1 | 100.63% | 100.00% | 99.80% | 100.00% | 22 |
| Tube2 | 10.26% | 10.37% | 10.73% | 10.00% | 26 |
| Tube3 | 4.83% | 4.83% | 5.49% | 5.00% | 26 |
| Tube4 | 1.10% | 1.08% | 1.88% | 1.00% | 27 |
| Tube5 | 0.53% | 0.49% | 1.16% | 0.50% | 29 |
| Tube6 | 0.33% | 0.18% | 1.49% | 0.10% | 18 |
| Tube7 | 0.18% | 0.03% | 1.02% | 0.00% | 21 |
| Tube8 | NaN | NA | NaN | None | 0 |
| Tube9 | 2.26% | 2.09% | 3.39% | 2.00% | 20 |
| Tube10 | 2.08% | 2.15% | 2.82% | 1.75% | 25 |
| Tube11 | 1.32% | 1.30% | 2.19% | 1.50% | 17 |
| Tube12 | 1.10% | 1.06% | 2.00% | 1.25% | 17 |
| Tube13 | 0.67% | 0.61% | 1.53% | 0.75% | 17 |
| Tube14 | 0.28% | 0.28% | 1.29% | 0.25% | 16 |
| Tube15 | 7.38% | 6.98% | 8.28% | 7.14% | 23 |

Again, the raw mean is strongly biased by individual outlier target values. The median was again closer in absolute value to the “intended percentage” than the other two candidate measures in nine of fourteen samples. The raw mean was closest in seven, and the trimmed was closest in zero. Overall the median was more accurate more often.

### Example 4—Examples of Computer-Implemented Embodiments

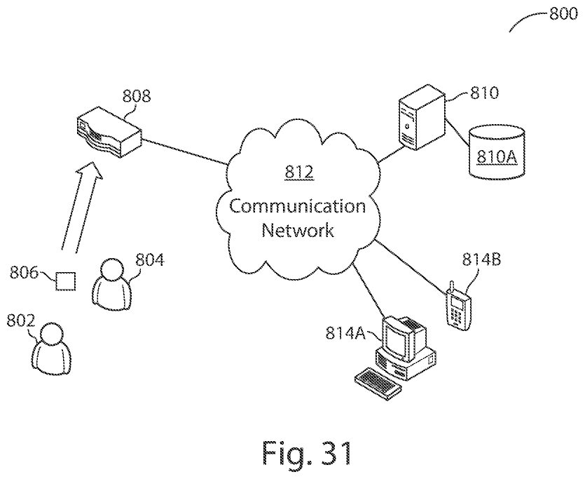

In some embodiments, the diagnostic techniques described above may be implemented via one or more computing devices executing one or more software facilities to analyze samples for a subject over time, measure cell-free nucleic acids (such as DNA) in the samples, and produce a diagnostic result based on one or more of the samples. FIG. 31 illustrates an example of a computer system with which some embodiments may operate, though it should be appreciated that embodiments are not limited to operating with a system of the type illustrated in FIG. 31.

The computer system of FIG. 31 includes a subject 802 and a clinician 804 that may obtain a sample 806 from the subject 806. As should be appreciated from the foregoing, the sample 806 may be any suitable sample of biological material for the subject 802 that may be used to measure the presence of cell-free nucleic acids (such as DNA) in the subject 802, including a blood sample. The sample 806 may be provided to an analysis device 808, which one of ordinary skill will appreciate from the foregoing will analyze the sample 808 so as to determine (including estimate) an amount of a non-native cell-free nucleic acids (such as DNA) in the sample 806 and/or the subject 802. For ease of illustration, the analysis device 808 is depicted as single device, but it should be appreciated that analysis device 808 may take any suitable form and may, in some embodiments, be implemented as multiple devices. To determine the amounts of cell-free nucleic acids (such as DNA) in the sample 806 and/or subject 802, the analysis device 808 may perform any of the techniques described above, and is not limited to performing any particular analysis. The analysis device 808 may include one or more processors to execute an analysis facility implemented in software, which may drive the processor(s) to operate other hardware and receive the results of tasks performed by the other hardware to determine on overall result of the analysis, which may be the amounts of cell-free nucleic acids (such as DNA) in the sample 806 and/or the subject 802. The analysis facility may be stored in one or more computer-readable storage media, such as a memory of the device 808. In other embodiments, techniques described herein for analyzing a sample may be partially or entirely implemented in one or more special-purpose computer components such as Application Specific Integrated Circuits (ASICs), or through any other suitable form of computer component that may take the place of a software implementation.

In some embodiments, the clinician 804 may directly provide the sample 806 to the analysis device 808 and may operate the device 808 in addition to obtaining the sample 806 from the subject 802, while in other embodiments the device 808 may be located geographically remote from the clinician 804 and subject 802 and the sample 806 may need to be shipped or otherwise transferred to a location of the analysis device 808. The sample 806 may in some embodiments be provided to the analysis device 808 together with (e.g., input via any suitable interface) an identifier for the sample 806 and/or the subject 802, for a date and/or time at which the sample 806 was obtained, or other information describing or identifying the sample 806.

The analysis device 808 may in some embodiments be configured to provide a result of the analysis performed on the sample 806 to a computing device 810, which may include a data store 810A that may be implemented as a database or other suitable data store. The computing device 810 may in some embodiments be implemented as one or more servers, including as one or more physical and/or virtual machines of a distributed computing platform such as a cloud service provider. In other embodiments, the device 810 may be implemented as a desktop or laptop personal computer, a smart mobile phone, a tablet computer, a special-purpose hardware device, or other computing device.

In some embodiments, the analysis device 808 may communicate the result of its analysis to the device 810 via one or more wired and/or wireless, local and/or wide-area computer communication networks, including the Internet. The result of the analysis may be communicated using any suitable protocol and may be communicated together with the information describing or identifying the sample 806, such as an identifier for the sample 806 and/or subject 802 or a date and/or time the sample 806 was obtained.

The computing device 810 may include one or more processors to execute a diagnostic facility implemented in software, which may drive the processor(s) to perform diagnostic techniques described herein. The diagnostic facility may be stored in one or more computer-readable storage media, such as a memory of the device 810. In other embodiments, techniques described herein for analyzing a sample may be partially or entirely implemented in one or more special-purpose computer components such as Application Specific Integrated Circuits (ASICs), or through any other suitable form of computer component that may take the place of a software implementation.

The diagnostic facility may receive the result of the analysis and the information describing or identifying the sample 806 and may store that information in the data store 810A. The information may be stored in the data store 810A in association with other information for the subject 802, such as in a case that information regarding prior samples for the subject 802 was previously received and stored by the diagnostic facility. The information regarding multiple samples may be associated using a common identifier, such as an identifier for the subject 802. In some cases, the data store 810A may include information for multiple different subjects.

The diagnostic facility may also be operated to analyze results of the analysis of one or more samples 806 for a particular subject 802, identified by user input, so as to determine a diagnosis for the subject 802. The diagnosis may be a conclusion of a risk that the subject 802 has, may have, or may in the future develop a particular condition. The diagnostic facility may determine the diagnosis using any of the various examples described above, including by comparing the amounts of cell-free nucleic acids (such as DNA) determined for a particular sample 806 to one or more thresholds or by comparing a change over time in the amounts of cell-free nucleic acids (such as DNA) determined for samples 806 over time to one or more thresholds. For example, the diagnostic facility may determine a risk to the subject 802 of a condition by comparing an amount of a non-native cell-free nucleic acids (such as DNA) for the same sample(s) 806 to another threshold. Based on the comparisons to the thresholds, the diagnostic facility may produce an output indicative of a risk to the subject 802 of a condition.

As should be appreciated from the foregoing, in some embodiments, the diagnostic facility may be configured with different thresholds to which amounts of cell-free nucleic acids (such as DNA) may be compared. The different thresholds may, for example, correspond to different demographic groups (age, gender, race, economic class, presence or absence of a particular procedure/condition/other in medical history, or other demographic categories), different conditions, and/or other parameters or combinations of parameters. In such embodiments, the diagnostic facility may be configured to select thresholds against which amounts of cell-free nucleic acids (such as DNA) are to be compared, with different thresholds stored in memory of the computing device 810. The selection may thus be based on demographic information for the subject 802 in embodiments in which thresholds differ based on demographic group, and in these cases demographic information for the subject 802 may be provided to the diagnostic facility or retrieved (from another computing device, or a data store that may be the same or different from the data store 810A, or from any other suitable source) by the diagnostic facility using an identifier for the subject 802. The selection may additionally or alternatively be based on the condition for which a risk is to be determined, and the diagnostic facility may prior to determining the risk receive as input a condition and use the condition to select the thresholds on which to base the determination of risk. It should be appreciated that the diagnostic facility is not limited to selecting thresholds in any particular manner, in embodiments in which multiple thresholds are supported.

In some embodiments, the diagnostic facility may be configured to output for presentation to a user a user interface that includes a diagnosis of a risk and/or a basis for the diagnosis for a subject 802. The basis for the diagnosis may include, for example, amounts of cell-free nucleic acids (such as DNA) detected in one or more samples 806 for a subject 802. In some embodiments, user interfaces may include any of the examples of results, values, amounts, graphs, etc. discussed above. They can include results, values, amounts, etc. over time. In some cases the graph may be annotated to indicate to a user how different regions of the graph may correspond to different diagnoses that may be produced from an analysis of data displayed in the graph. For example, thresholds against which the graphed data may be compared to determine the analysis may be imposed on the graph(s). This may include adding lines to the graph, separating the graph into sections, etc. In some embodiments, the sections may additionally or alternatively be shaded, such as with shading of different transparencies and/or colors. In embodiments in which the diagnostic facility evaluates more than two thresholds, more areas may be indicated through lines and/or shading.

A user interface, particularly with the lines and/or shading, may provide a user with a far more intuitive and faster-to-review interface to determine a risk of the subject 802 based on amounts of cell-free nucleic acids (such as DNA), than may be provided through other user interfaces. As such, there may be specific and substantial benefit to a user interface as provided herein. A user interface, particularly with the lines and/or shading, may also provide a user with a far more intuitive and faster-to-review interface to determine a risk of the subject 802 based on amounts of cell-free nucleic acids (such as DNA), than may be provided through other user interfaces. It should be appreciated, however, that embodiments are not limited to being implemented with any particular user interface.

In some embodiments, the diagnostic facility may output the diagnosis or a user interface to one or more other computing devices 814 (including devices 814A, 814B) that may be operated by the subject 802 and/or a clinician, which may be the clinician 804 or another clinician. The diagnostic facility may transmit the diagnosis and/or user interface to the device 814 via the network(s) 812.

Techniques operating according to the principles described herein may be implemented in any suitable manner. Included in the discussion above are a series of flow charts showing the steps and acts of various processes that determine a risk of a condition based on an analysis of amounts of cell-free nucleic acids (such as DNA). The processing and decision blocks discussed above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that embodiments are not limited to any particular syntax or operation of any particular circuit or of any particular programming language or type of programming language. Rather, one skilled in the art may use the description above to fabricate circuits or to implement computer software algorithms to perform the processing of a particular apparatus carrying out the types of techniques described herein. It should also be appreciated that, unless otherwise indicated herein, the particular sequence of steps and/or acts described above is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionality may be implemented in a single functional facility. It should also be appreciated that, in some implementations, some of the functional facilities described herein may be implemented together with or separately from others (i.e., as a single unit or separate units), or some of these functional facilities may not be implemented.

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as a portion of a computing device or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, including the exemplary computer system of FIG. 31, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing device (co-located or geographically distributed) dedicated to executing the techniques described herein, one or more Field-Programmable Gate Arrays (FPGAs) for carrying out the techniques described herein, or any other suitable system.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Any one of the aforementioned, including the aforementioned devices, systems, embodiments, methods, techniques, algorithms, media, hardware, software, interfaces, processors, displays, networks, inputs, outputs or any combination thereof are provided herein in other aspects.

### Example 5—Exemplary Assays

Genotyping

A multiplexed, allele-specific quantitative PCR-based assay can be used to calculate donor fraction (DF) as a percentage of cf-DNA. A panel of high frequency SNPs are selected for their ability to reliably discriminate between alleles. Briefly, 15 ng of total cf-DNA is added to a multiplexed library master mixture with an exogenous standard spiked into each sample (4.5E+03 copies) and amplified by PCR for 35 cycles in a 25 ul reaction containing 0.005 U Q5 (NEB) DNA polymerase, 0.2 mM dNTPs, 3 uM forward primer pool of 96 targets, 3 uM reverse primer pool of 96 targets, at a final concentration of 2 mM MgCl2.

Cycling conditions can be 98° C. for 30 s, then 35 cycles of 98° C. for 10 s, 55° C. for 40 s, and 72° C. for 30 s. This can then be finished with a 2-minute incubation at 72° C. and then stored at 4° C. Ten microliters of the final reaction is cleaned up with ExoSAP-IT (Thermo Fisher Scientific) by incubating at 37° C. for 15 minutes followed by 80° C. for 15 minutes. Libraries are then diluted with Preservation Buffer and either processed for genotyping or stored at −80° C. Quantitative genotyping (qGT) is performed starting from 3 8 ul of a 1:100 dilution of the preserved library diluted 1:100 and run in duplicate 3 ul reactions with appropriate controls and calibrators on the Roche LightCycler 480 platform (Roche Diagnostics, Indianapolis, Ind.). A procedure is used to assign the genomic DNA (gDNA) of the recipient or donor with one of three possible genotypes at each target loci (i.e. homozygous AA, heterozygous AB and homozygous BB).

Donor Fraction (Specific) Analysis

Standard curves of heterozygous DNA sources are used to quantify alleles at each target. Quality control procedures can be used to evaluate each standard curve and sample amplification. Quantifiable targets can proceed to interpretation. Acceptability criteria can include historic amplification shape, specificity of the allele specific PCR assay with respect to the second allele, signal to noise, slope and r-squared of standard curve sets, amplification of controls, and contamination of negative controls.

With the labels of recipient and/or donor possible genotypes at each target (e.g. homozygous AA, heterozygous AB, and homozygous BB, informative targets can be defined as those where the recipient is known homozygous and the donor has a different genotype. Where the donor is homozygous and different from the recipient the target is referred to as fully-informative, because the observed B allele ratio is approximately the overall DF level. Where the donor is heterozygous the target is called half-informative because the contribution is to both the A and B alleles, and the measured contribution is doubled. The median of informative and quality-control-passed allele ratios is calculated and reported as DF (%) of total cf-DNA.

Each quantitative genotyping process can yield two quality control measures, the rCV and dQC. The regularized robust coefficient of variation (rCV) is computed using the distribution of the informative and quantifiable targets. First the robust standard deviation (rSD) is computed as the median absolute divergence from the median minor species proportion. The rSD is converted to a coefficient of variation by dividing by the median after it has been regularized. The rCV measures the spread of assayed targets around their median and can serve as a metric of precision or sample quality. The dQC is a discordance quality check, such as an evaluation of the average minor allele proportion of recipient homozygous and non-informative targets (can be performed as a safeguard against contamination.)

1. A method of assessing an amount of non-native nucleic acids in a sample from a subject, the sample comprising non-native and native nucleic acids, the method comprising:
obtaining results from a mismatch amplification-based quantification assay, and
determining an amount of the non-native nucleic acids in the sample based on the results, wherein the determining comprises averaging the results to determine the amount, and the averaging is taking the median.

2. The method of claim 1, wherein the determining comprises or the method further comprises analyzing the results using a robust standard deviation and/or robust coefficient of variation.

3. The method of claim 1 or 2, wherein the determining comprises or the method further comprises analyzing the results using a discordance value.

4. A method of assessing an amount of non-native nucleic acids in a sample from a subject, the sample comprising non-native and native nucleic acids, the method comprising:
obtaining results from a mismatch amplification-based quantification assay, and
determining an amount of the non-native nucleic acids in the sample based on the results, wherein the determining comprises analyzing the results using a robust standard deviation and/or robust coefficient of variation.

5. The method of claim 4, wherein the determining comprises or the method further comprises analyzing the results using a discordance value.

6. A method of assessing an amount of non-native nucleic acids in a sample from a subject, the sample comprising non-native and native nucleic acids, the method comprising:
obtaining results from a mismatch amplification-based quantification assay, and
determining an amount of the non-native nucleic acids in the sample based on the results, wherein the determining comprises analyzing the results using a discordance value.

7. The method of any one of the preceding claims, wherein the amount is provided in a report.

8. A method of assessing a risk in a subject based on one or more amounts of non-native nucleic acids in one or more samples from a subject, the sample(s) comprising non-native and native nucleic acids, the method comprising:
obtaining one or more amounts of non-native nucleic acids in one or more samples from a subject, which amounts are determined from the results of one or more mismatch amplification-based quantification assays, and
assessing a risk based on the amount(s) of non-native nucleic acids.

9. The method of claim 8, wherein the amount(s) are obtained from a report.

10. The method of any one of the preceding claims, wherein the amount(s) is the ratio or percentage of non-native nucleic acids to native nucleic acids or total nucleic acids.

11. The method of claim 10, wherein the amount of the native or total nucleic acids is also determined.

12. The method of any one of the preceding claims, wherein each mismatch amplification-based quantitative assay comprises:
for each of a plurality of single nucleotide variant (SNV) targets, performing amplification on the nucleic acids of the sample, or portion thereof, with at least two primer pairs, wherein each primer pair comprises a forward primer and a reverse primer, wherein one of the at least two primer pairs comprises a 3′ penultimate mismatch in a primer relative to one allele of the SNV target but a 3′ double mismatch relative to another allele of the SNV target and specifically amplifies the one allele of the SNV target, and another of the at least two primer pairs specifically amplifies the another allele of the SNV target, and
and obtaining or providing results from the amplifications.

13. The method of claim 12, wherein the another primer pair of the at least two primer pairs also comprises a 3′ penultimate mismatch relative to the another allele of the SNV target but a 3′ double mismatch relative to the one allele of the SNV target in a primer and specifically amplifies the another allele of the SNV target.

14. The method of claim 12 or 13, wherein the results are informative results of the amplifications.

15. The method of any one of claims 12-14, wherein the mismatch amplification-based quantitative assay further comprises selecting informative results of the amplification assays.

16. The method of any one of claims 12-15, wherein the informative results of the amplifications are selected based on the genotype of the non-native nucleic acids and/or native nucleic acids.

17. The method of any one of claims 12-16, wherein the mismatch amplification-based quantitative assay further comprises obtaining the genotype of the non-native nucleic acids and/or native nucleic acids.

18. The method of any one of claims 12-17, wherein the mismatch amplification-based quantitative assay further comprises obtaining the plurality of SNV targets.

19. The method of any one of claims 12-18, wherein the mismatch amplification-based quantitative assay further comprises obtaining the at least two primer pairs for each of the plurality of SNV targets.

20. The method of any one of claims 12-19, wherein the plurality of SNV targets is at least 90 SNV targets.

21. The method of claim 20, wherein the plurality of SNV targets is at least 95 SNV targets.

22. The method of claim 20 or 21, wherein the plurality of SNV targets is less than 105 SNV targets.

23. The method of claim 22, wherein the plurality of SNV targets is less than 100 SNV targets.

24. The method of any one of claims 12-23, wherein when the genotype of the non-native nucleic acids is not known or obtained, the mismatch amplification-based quantitative assay further comprises:
assessing results based on a prediction of the likely non-native genotype.

25. The method of claim 24, wherein the assessing is performed with an expectation-maximization algorithm.

26. The method of any one of claims 12-25, wherein the mismatch amplification-based quantitative assay further comprises selecting informative results based on the native genotype and prediction of the likely non-native genotype.

27. The method of claim 26, wherein expectation-maximization is used to predict the likely non-native genotype.

28. The method of any one of claims 12-27, wherein the mismatch amplification-based quantitative assay further comprises obtaining the genotype of the native nucleic acids.

29. The method of any one of claims 12-28, wherein the mismatch amplification-based quantitative assay further comprises obtaining the plurality of SNV targets.

30. The method of any one of claims 12-29, wherein the mismatch amplification-based quantitative assay further comprises obtaining the at least two primer pairs for each of the plurality of SNV targets.

31. The method of any one of claims 12-30, wherein maximum likelihood is used to determine the amount of non-native nucleic acids.

32. The method of any one of the preceding claims, wherein the sample(s) comprise cell-free DNA sample and the amount is an amount of non-native cell-free DNA.

33. The method of any one of the preceding claims, wherein the subject is a transplant recipient, and the amount of non-native nucleic acids is an amount of donor-specific cell-free DNA.

34. The method of claim 33, wherein the transplant recipient is a heart transplant recipient.

35. The method of claim 33 or 34, wherein the transplant recipient is a pediatric transplant recipient.

36. The method of any one of claim 12-35, wherein the amplifications are by quantitative PCR, such as real time PCR or digital PCR.

37. The method of any one of claims 1-7 and 9-36, wherein the method further comprises determining a risk based on the amount(s).

38. The method of claim 8 or 37, wherein the risk is a risk associated with a transplant.

39. The method of claim 38, wherein the transplant is a heart transplant.

40. The method of claim 38 or 39, wherein the transplant is a pediatric transplant.

41. The method of any one of the preceding claims, wherein the method further comprises or the assessing comprises selecting a treatment for the subject based on the amount(s) of non-native nucleic acids.

42. The method of any one of the preceding claims, wherein the method further comprises or the assessing comprises treating the subject based on the amount(s) of non-native nucleic acids.

43. The method of any one of the preceding claims, wherein the method further comprises or the assessing comprises providing information about a treatment to the subject based on the amount(s) of non-native nucleic acids.

44. The method of any one of the preceding claims, wherein the method further comprises or the assessing comprises monitoring or suggesting the monitoring of the amount(s) of non-native nucleic acids in the subject over time.

45. The method of any one of the preceding claims, wherein the method further comprises or the assessing comprises obtaining the amount(s) of non-native nucleic acids in the subject at a subsequent point in time.

46. The method of any one of the preceding claims, wherein the method further comprises or the assessing comprises evaluating an effect of a treatment administered to the subject based on the amount(s) of non-native nucleic acids.

47. The method of any one of claims 41-43 and 46, wherein the treatment is an anti-rejection therapy.

48. The method of any one of claims 41-43 and 46, wherein the treatment is an anti-infection therapy.

49. The method of any one of the preceding claims, further comprising providing or obtaining the sample(s) or a portion thereof.

50. The method of any one of the preceding claims, further comprising extracting nucleic acids from the sample(s).

51. The method of any one of the preceding claims, wherein the sample(s) comprise blood, plasma or serum.

52. The method of any one of the preceding claims, wherein the sample(s) are from the subject within 10 days of a transplant, such as a heart transplant.

53. The method of any one of the preceding claims, wherein the sample(s) are from the subject within 24 hours of a transplant, such as a heart transplant.

54. The method of any one of the preceding claims, wherein the sample(s) are from the subject within 24 hours of cross-claim removal, such as in a heart transplant.
